(19) United States
(12) Patent Application Publication
(10) Pub. No.: US 2008/0274156 A1
(43) Pub. Date: Nov. 6, 2008

(54) Composition forstimulating bone growth and differentiation and method for isolating same

(75) Inventors: Victor Nurcombe, Proteos (SG); Simon Cool, Proteos (SG)

Correspondence Address: STOUT, UXA, BUYAN & MULLINS LLP, 4 VENTURE, SUITE 300, IRVINE, CA, 92618, US

(73) Assignee: The University of Queensland, Brisbane (AU)

(21) Appl. No.: 12/012,860

(22) Filed: Feb. 5, 2008

Related U.S. Application Data
(62) Division of application No. 11/123,897, filed on May 6, 2005.

(51) Int. Cl. A61K 31/727; A61F 2/28

(52) U.S. Cl. 424/423; 514/56

(57) ABSTRACT

This invention relates to isolated heparan sulphate and use thereof to stimulate bone cell growth and differentiation. The invention also relates to use of heparan sulphate with implants, prosthesis and bioscaffolds to repair and regenerate bone. Such use may be for repair of damaged tissue including bone tissue, for example damage resulting from injury or defect.

This application is a divisional of U.S. patent application Ser. No. 11/123,897, filed May 6, 2005, which claims priority of Australian Provisional Application Ser. No 2004902408, filed May 7, 2004.

### FIELD OF THE INVENTION

This invention relates to isolated heparan sulphate and use thereof to stimulate bone cell growth and differentiation. The invention also relates to use of heparan sulphate with implants, prosthesis and bioscaffolds to repair and regenerate bone. Such use may be for repair of damaged tissue including bone tissue, for example damage resulting from injury or defect.

### BACKGROUND OF THE INVENTION

Although there have been advances in the area of implants anchored in bone tissue, current orthopaedic practices for repair of load-bearing bone are rather crude and often fail. Use of titanium-based materials has improved implants; however, there is still a need for methods and compositions that may assist or stimulate regeneration of natural bone for use with, or independently from, an implant.

Humans and other animals are complex multicellular organisms that control tissue repair using a number of mechanisms, including for example, cell differentiation, cell propagation, migration, growth, cell-to-cell and cell-to-substrate interactions. Many of these mechanisms are under the control of extracellular mediators or cytokines, including growth factors.

Heparan sulphate (HS) glycosaminoglycans, located immediately adjacent to the surfaces of neighbouring cells, modulate the action of a large number of extracellular ligands, including growth factors. It does this with a complicated combination of autocrine, juxtacrine and paracrine feedback loops. HS are essential regulators of fibroblast growth factor (FGF) activity both in vivo and in vitro, and function by cross-linking particular forms of FGF to appropriate FGF receptors. Although HS may be generically described (Rabenstein, 2002, Nat. Prod. Rep. 19 (3) 312; incorporate herein by reference; see also FIG. 1), HS species isolated from a single source may differ in biological activity. As shown in Brickman et al, 1998, Glycobiology 8 463, two separate pools of HS obtained from neuroepithelial cells could specifically activate either FGF-1 or FGF-2, depending on mitogenic status. HS isolated from log growth phase cells potentiated FGF-2 activity, and HS isolated from contact-inhibited cells preferentially activated FGF-1.

A HS that is capable of interacting with either FGF-1 or FGF-2 is described in WO 96/23003. According to this patent application, a respective HS capable of interacting with FGF-1 is obtainable from murine cells at embryonic day from about 11 to about 13, whereas a HS capable of interacting with FGF-2 is obtainable at embryonic day from about 8 to about 10.

HS is usually secreted from a cell coupled to a protein core, and is thus referred to as a heparan sulphate proteoglycan (HSPG). Both HS and core protein may undergo a series of modifications that may ultimately influence their biological activity. Complexity of HS has been considered to surpass that of nucleic acids (Lindahl et al, 1998, J. Biol. Chem. 273 24979; Sugahara and Kitagawa, 2000, Curr. Opin. Struct. Biol. 10 518).

Use of a polysaccharide, including HS, in combination with chitosan has been described in International patent application WO 96/02259 for manufacture of an agent capable of stimulating regeneration of hard tissue. No examples specifically describing how HS is to be used, nor a source or type of HS are described in the patent application. In addition, a composition for stimulating de novo bone induction is disclosed in WO 03/079964. In one embodiment, a reconstituted basement membrane of this composition may optionally contain HS. The composition further contains a bone morphogenetic protein, as well as optionally (typically as the morphogenetic protein) an osteogenic protein and a transformation growth factor.

International patent application WO 93/19096 describes oligosaccharides obtained from HS from confluent cultures of human skin fibroblasts having growth factor binding activity, for example FGF or HS-protein binding affinity. The oligosaccharides are described as being useful as therapeutics for blocking cell surface signal transduction and inhibiting growth factor activity. The oligosaccharides are particularly useful due to their minimal size and specific binding affinity.

WO 93/19096 states that in contrast to the useful properties of oligosaccharides as therapeutics, HS is not particularly useful as a therapeutic. In fact, even fragments of HS (i.e. oligosaccharides prepared from enzyme digested HS), are also not considered suitable for use as a therapeutic due to a resulting complex mixture of various molecular species having a wide range of different compositions and sizes. Accordingly, WO 93/19096 advises against use of HS, or enzyme digested preparations thereof, for use as a therapeutic.

### SUMMARY OF THE INVENTION

However, contrary to WO 93/19096, the present inventors have surprising found that HS obtained from a specific tissue source may have particularly useful properties, in particular as a potential therapeutic and pharmaceutical composition. Although HS has been previously extracted from skin, brain, liver and cultured cells, HS has never been extracted from bone or bone precursor cells prior to this invention. The inventors were surprised to find that HS isolated from bone cells when applied to cells showed a greater increase in bone cell growth when compared with other sources of HS, as is described in more detail hereinafter.

In a first aspect, the invention provides isolated heparan sulphate obtained from bone, bone cell, bone precursor cell or stem cell

In one embodiment, the bone, bone cell, bone precursor cell or stem cell is obtained from a mammal.

In some embodiments the mammal is a human, bovine, pig or rodent.

As an example, the mammal may be a human.

In one embodiment, the bone cell, bone precursor cell or stem cell is cultured.

In some embodiments, the bone cell, bone precursor cell or stem cell is isolated and cultured to remove other cell types.

As an example, the bone precursor cell may be selected from the group consisting of KS-4, UMR106, UMR201, MBA 15.4, 2T3, and MC3T3-E1.

The HS may be isolated from cultured cells either during logarithmic growth phase or when contact inhibited.

Preferably, the HS is isolated from cultured cells during logarithmic growth phase.

In a second aspect, the invention provides a method for isolating heparan sulphate including the step of purifying heparan sulphate from a tissue or cell selected from the group consisting of: bone, bone cell, bone precursor cell and stem cell.

In one embodiment, the bone, bone cell, bone precursor cell or stem cell is obtained from a mammal.

In some embodiments the mammal is a human, bovine, pig or rodent.

In one embodiment, the bone cell, bone precursor cell or stem cell is cultured.

As an example, the bone precursor cell may be selected from the group consisting of KS-4, UMR106, UMR201, MBA 15.4, 2T3, and MC3T3-E1.

The HS may be isolated from cultured cells either during logarithmic growth phase or when contact inhibited.

Preferably, the HS is isolated from cultured cells during logarithmic growth phase.

In a third aspect, the invention provides isolated heparan sulphate obtainable according to the method of the second aspect.

In a fourth aspect, the invention provides a pharmaceutical composition comprising isolated heparan sulphate according to the first and third aspects in combination with a carrier or diluent.

In one embodiment, the pharmaceutical composition further comprises one or more biologically active molecule(s) capable of stimulating bone or bone cell growth and/or differentiation.

In one embodiment, the one or more biologically active molecule(s) is selected from the group consisting of: BMP2, BMP4, OP-1, FGF1, FGF2, TGF-β1, TGF-β2, TGF-β3, Collagen 1, laminin 1-6, fibronectin and vitronectin.

The composition may further comprise one or more bis-phosphonates.

In one embodiment the bis-phosphonate is selected from the group consisting of: etidronate, clodronate, alendronate, pamidronate, risedronate and zoledronate.

The pharmaceutical composition may be used in the manufacture of a medicament for treating an animal in need of tissue repair.

The tissue to be repaired may be soft or hard tissue.

In one embodiment the tissue to be repaired is hard tissue.

In one embodiment the hard tissue is bone.

In one embodiment the repair of the hard tissue comprises a step of administering the pharmaceutical composition by coating or impregnating a surgical implant, prosthesis or bioscaffold before implantation.

In one embodiment, the animal is a mammal.

In one embodiment, the mammal is a human, bovine, pig or rodent.

In one embodiment, the mammal is thus a human.

In a fifth aspect, the invention provides a surgical implant, prosthesis or bioscaffold comprising isolated heparan sulphate according to the first and third aspects.

In one embodiment, the surgical implant, prosthesis or bioscaffold is coated or impregnated with the isolated heparan sulphate.

The surgical implant, prosthesis or bioscaffold may be further coated or impregnated with BMP2, BMP4, OP-1, FGF1, FGF2, TGF-β1, TGF-β2, TGF-β3, Collagen 1, laminin 1-6, fibronectin and vitronectin.

The surgical implant, prosthesis or bioscaffold may be still further coated or impregnated with etidronate, clodronate, alendronate, pamidronate, risedronate and zoledronate.

In one embodiment, the bioscaffold comprises a polymer that incorporates either hydroxyapatite or hyaluronic acid.

The surgical implant, prosthetic or bioscaffold may be used with hard tissue.

In one embodiment, the hard tissue is bone.

In one embodiment, the surgical implant, prosthesis or bioscaffold is used to repair dental damage.

In a sixth aspect, the invention provides a method of treating an animal in need of tissue repair including the steps of administering a pharmaceutical composition of the fourth aspect.

The tissue to be repaired may be soft or hard tissue.

In one embodiment, the tissue is hard tissue.

In one embodiment, the hard tissue is bone.

In one embodiment, repair of the hard tissue includes the step administering the pharmaceutical composition by coating or impregnating a surgical implant, prosthesis or bioscaffold of the fifth aspect before implantation.

In one embodiment, the animal is a mammal.

In one embodiment, the mammal is a human, bovine, pig or rodent.

In one embodiment, the mammal is thus a human.

In a seventh aspect, the invention provides use of the isolated heparan sulphate of the first or third aspect for stimulating regeneration of tissue.

In this aspect the invention also provides use of the isolated heparan sulphate of the first or third aspect in the manufacture of a medicament for stimulating regeneration of tissue

The tissue may be soft or hard tissue.

In one embodiment, the tissue is hard tissue.

In one embodiment, the hard tissue is bone.

In an eighth aspect, the invention provides a process for stimulating regeneration of tissue including the step of applying the isolated heparan sulphate of the first or third aspects to an area of a body in need of tissue regeneration.

The tissue may be soft or hard tissue.

In one embodiment, the tissue is hard tissue.

In one embodiment, the hard tissue is bone.

In a ninth aspect, the invention provides use of the isolated heparan sulphate of the first or third aspect for stimulating differentiation of a cell into a bone or bone-like cell.

In one embodiment, the cell is a stem cell.

In one embodiment, the stem cell is an embryonic stem cell.

One or more biologically active molecule(s) capable of stimulating bone or bone cell growth and/or differentiation may also be added to the cell in addition to the isolated heparan sulphate.

In one embodiment, the one or more biologically active molecule(s) is selected from the group consisting of: BMP2, BMP4, OP-1, FGF1, FGF2, TGF-β1, TGF-β2, TGF-β3, Collagen 1, laminin 1-6, fibronectin and vitronectin

One or more bis-phosphonates may also be added to the cell.

In one embodiment, the bis-phosphonate is selected from the group consisting of: etidronate, clodronate, alendronate, pamidronate, risedronate and zoledronate.

In a tenth aspect, the invention provides a method for identifying a biologically active molecule including the step of determining whether one or more candidate molecule(s) binds to the isolated heparan sulphate of the first or third aspects.

In one embodiment, the method further includes the step of determining a biological function of said molecule.

In one embodiment, the biologically active molecule is capable of stimulating bone or bone cell growth and/or differentiation.

The candidate molecule may be a natural or synthetic molecule; an extract from a plant or animal, tissue or cell; a product from a recombinatorial library, cDNA library or expression library; a drug or chemical; carbohydrate; or protein.

In one embodiment, the protein is a growth factor.

Throughout this specification unless the context requires otherwise, the word “comprise”, and variations such as “comprises” or “comprising”, will be understood to imply the inclusion of the stated integers or group of integers or steps but not the exclusion of any other integer or group of integers.

### DESCRIPTION OF THE FIGURES AND TABLES

In order that the invention may be readily understood and put into practical effect, exemplary embodiments will now be described by way of illustrative example with reference to the accompanying drawings wherein like reference numerals refer to like parts.

FIG. 1 schematically depicts the structural composition of heparan sulphate (HS). HS GAG sugars comprise repeating disaccharide units of amino sugars linked to uronic acid that are varied in sulphation, where R′═H or SO₃═ and R″═COCH₃, SO₃— or H.

FIG. 2 shows the metabolic activity of MC3T3-E1 cells during the initial growth phase, seeded at 10 000 cells/cm². Metabolic activity was measured by the conversion of WST-1 to formazan by mitochondrial dehydrogenase. This conversion liberates a red colour that is measured at 450 nm with a reference wavelength of 630 nm. The absorbance directly correlates to the proportion of metabolically-active cells in the culture. These results are the mean±standard deviation of three independent repeats, each repeat conducted in triplicate.

FIG. 3 depicts the metabolic activity of MC3T3-E1 cells seeded at 5 000 cells/cm². Metabolic activity was measured using WST-1 as in FIG. 2. The comparison with FIG. 2 illustrates that MC3T3-E1 cells are stably growing at different cell densities.

FIG. 4 depicts the proliferation of MC3T3-E1 cells seeded at 2500 cells/cm² over a period of 20 days. Proliferation was measured using BrdU incorporation and the results are displayed as the mean±SD. As proliferation depends on metabolic activity, a corresponding metabolic activity can be implied.

FIG. 5 depicts the differentiation status characterized by the expression of marker proteins.

FIG. 6 shows an elution profile of recovery of HS from a DEAE ion exchange column.

FIG. 7 shows elution profiles from a Sepharose CL-6B column to separate HS chains and fragments.

FIG. 8 shows elution profiles of gel filtration on Bio-Gel P-10 of oligosaccharides produced by depolymerising agents: (A) low pH HNO2 (B) heparitinase and (C) heparinase.

FIG. 9 shows an elution profile of strong anion exchange-high pressure liquid chromatography (SAX-HPLC) of disaccharides produced by complete glycosaminoglycan lyase depolymerisation.

FIG. 10 shows an elution profile of SAX-HPLC of HNO₂ generated disaccharides.

FIG. 11 shows a “finger print” of an HS disaccharide total profile/library by SAX-HPLC.

FIG. 12 shows a graph of effects of FGF-1 (black bars) and FGF-2 (white bars) on proliferation of MC3T3-E1 bone cells. Cell proliferation was monitored by BrdU incorporation.

FIG. 13 shows a graph of effects of bone-derived and non-bone-derived HS supplementation on proliferation of MC3T3-E1 bone cells.

FIG. 14 depicts a graph of effects of bone-derived and non-bone-derived HS supplementation from a different species on proliferation of osteoblasts. Human HS (hHS) and porcine HS (pHS) was added to pig osteoblasts (pig HOst) and human osteoblasts (hOst).

FIG. 15 depicts a dose-response curve of HS on proliferation of MC3T3-E1 bone cells. HS harvested from MC3T3-E1 cells demonstrated a concentration-dependent increase in proliferation when dosed back on immature MC3T3-E1 for 24 h. ED₅₀ was determined to be 5 μg/ml HS.

FIG. 16 illustrates the acceleration of the healing process of a bone fracture by HS. HS (5 or 50 μg) was delivered in a gel carrier into a mid-diaphyseal fracture in the femora of rats. Gel carrier alone was used as control. Radiographs were taken in the AP plane at 2 and 5 weeks post-surgery to determine the degree of healing across the fracture.

FIG. 17 depicts a von Kossa staining. Medial halves of treated femurs at both 2 and 5 weeks were sectioned and stained using von Kossa staining to show differences in callus mineralization between the 3 groups. Scale=400 μm.

FIG. 18 depicts the histomorphometric measurements for von Kossa stained sections. The graphs represent the mean±SD of the callus trabecular bone (BV/TV) (A), trabecular thickness ({BV×2}/TV) (B) and trabecular number ({BP×0.5}/TV×1000) (C), where BV=bone volume, TV=total volume and BP=bone perimeter. Black bars=control, grey bars=5 μg HS, and white bars=50 μg HS. Significant values are represented as *p<0.05 compared to controls.

FIG. 19 depicts the cartilage formation as determined by safranin O staining. Lateral halves of treated femurs were embedded in paraffin, sectioned and stained with safranin O and counter-stained with light green to distinguish the cartilage and bone respectively. Black bars=control, grey bars=5 μg HS, and white bars=50 μg HS.

FIG. 20 illustrates the specificity of the effect of HS. Osteoclast numbers were counted from 9 fields of view using a grid method for each of the 3 groups, (n=8 per group). The values displayed are the mean±SD. Black bars=control, grey bars=5 μg HS, and white bars=50 μg HS.

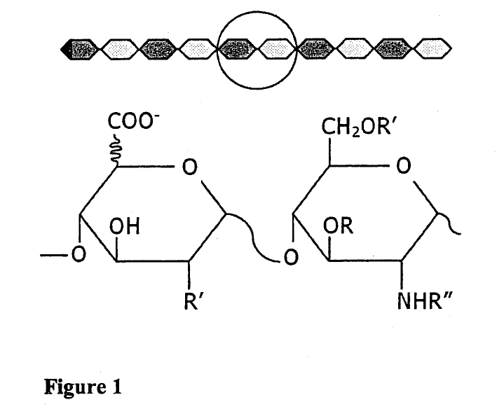

Table I depicts the disaccharide composition of HS as determined by SAX-HPLC following complete depolymerisation with HNO₂. Disaccharides had been isolated on a 1×120 cm Bio-Gel P-2 column (nd=not detected).

Table II depicts the disaccharide composition of HS as determined by SAX-HPLC. Heparansulphate had been isolated and completely depolymerised with a mixture of glycosaminoglycan-specific lyases. The resulting unsaturated disaccharides were isolated on a P-2 column and fractionated by strong anion exchange column chromatography. Numbers represent the average of three runs for samples. Over 97% disaccharides were recovered from each sample.

Table III depicts the comparative disaccharide compositions of the adenoma and carcinoma HS species.

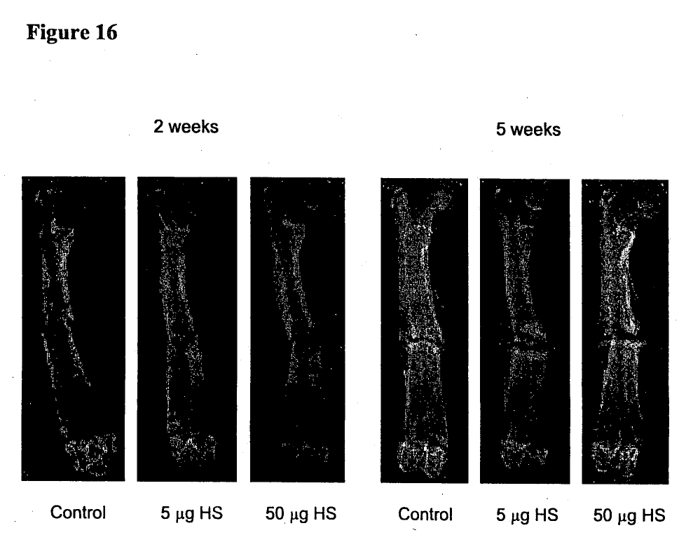

Table IV depicts the callus size for fractured femora at 2 and 5 weeks (see also FIG. 16). Values represent the anterior-posterior dimension (AP, mm) and the lateral dimension (Lat, mm). Data are the mean±SD values, *p<0.05 vs. control. ANOVA LSD post hoc.

### DETAILED DESCRIPTION OF THE INVENTION

In one embodiment of the invention the heparan sulphate glycosaminoglycan of the present invention is obtained from bone, bone cell, bone precursor cell or stem cell. Any source of bone, bone cell, bone precursor cell or stem cell may be used. In one embodiment, the bone, bone cell, bone precursor cell or stem cell is obtained from a mammal. Examples of a mammal, from which the bone, bone cell, bone precursor cell or stem cell may be obtained, include, but are not limited to, a human, bovine, a pig or a rodent. Examples of suitable rodents include, but are not limited to, a mouse, a rat or a guinea pig. The heparan sulphate may thus for example be obtained from a human.

In one embodiment, the bone cell, bone precursor cell or stem cell is cultured. In some embodiments, the bone cell, bone precursor cell or stem cell is isolated and cultured to remove other cell types. In other embodiments an available bone precursor cell line is used. Examples of suitable bone precursor cell lines include, but are not limited to, KS-4, UMR106, UMR201, MBA 15.4, 2T3, and MC3T3-E1.

The HS may be isolated from cultured cells either during logarithmic growth phase or when contact inhibited. In a preferred embodiment, the HS is isolated from cultured cells during logarithmic growth phase. In embodiments, where the cultured cells are MC3T3-E1 cells, the HS is typically isolated from cells of day 6-8 in culture, for example at day 7.

Heparan sulphate prepared in accordance with the invention may be used to direct a phenotypic change of a stem cell and/or bone precursor cell into a mature bone cell capable of engineering new bone. The novel heparan sulphate obtained from bone cell, bone precursor cell or stem cells is capable of directing stem cell phenotype. When coated onto an appropriate surface, the heparan sulphate of the invention triggers, then accelerates, then controls, growth and tissue-specific repair by stem cells. This process leads to engineering of new bone tissue. The new bone tissue typically has complete functionality and biomechanical properties, indistinguishable from normal bone.

The isolated bone-derived HS may be used for control of bone growth and repair processes. Bone-derived HS may be capable of greater stimulation of bone regeneration when compared with HS isolated from non-bone derived sources, such as neuroepithelial cells, because bone-derived HS is isolated from a tissue or cell source where it may ultimately be applied. Accordingly, use of bone-derived HS may favour differentiation of precursor or stem cells into bone cells when compared with other HS sources. For example, when bone-derived HS is applied to brain precursor cells, the cells begin changing into bone-like cells. Similarly, brain precursor cell derived HS changes bone marrow stem cells into neuron-like cells. Accordingly, a specific tissue derived HS may couple to a surface of a cell whereby extracellular influences pre-dispose the cells to change to the tissue from where the HS is obtained.

MC3T3-E1 cells, grown in the presence of sodium chlorate (an inhibitor of heparan sulphate chain assembly), show a time-dependent decrease in cell numbers, indicative of apoptosis. When excess bone-specific HS is added back to these cultures, the excess HS overcomes this inhibition thereby alleviating cell death. This finding indicates that growth of bone cells is dependent on their endogenous HS chains. Non-specific HS (i.e. not from bone tissue) does not replicate this alleviation of death. This provides yet additional support for the contention that bone-derived HS is a crucial regulator of bone phenotype.

In one embodiment of the invention, tissue or cells are isolated from an individual, the tissue or cells are cultured and propagated, HS are isolated from the tissue or cells and the isolated HS administered to the same (autologous) or different (heterologous) individual. Heterologous isolation and application of HS includes both individuals of a same species, for example human-to-human and individuals of different species, for example a human recipient and a bovine donor source. HS in accordance with the invention can be used with both hard and soft tissue repair.

Cells may also be selected from the group consisting of KS-4, UMR106, UMR201, MBA 15.4, 2T3, and MC3T3-E1.

The inventors have furthermore identified a growth phase of bone cells in culture and found it advantageous to isolate HS from bone cells during such growth phase (see below).

In another embodiment, HS in accordance with the invention may be used for changing stem cells, for example embryonic stem cells, into bone or bone-like cells.

It will be appreciated by one skilled in the art that HS comprises multiple different forms of HS, each potentially having distinct biological activity. For example, HS may comprise a different number of repeating distinct disaccharide units, wherein each disaccharide unit may comprise a sulphate group located at different positions on a disaccharide unit. Regions of a HS chain may comprise different “hot spots” characterised by binding a particular ligand, for example FGF-1 and/or FGF-2. Accordingly, one HS form may bind different ligand(s) than another form. HS of the present invention is known to at least bind collagen type 1, which is known to be prevalent in bone tissue.

Bone cell derived isolated HS of the invention comprises a unique composition when compared to HS isolate from other sources. For example, disaccharide composition of HS as determined by SAX-HPLC following complete depolymerisation with HNO₂ (Table I) or a mixture of lyases (Table II) is unique to bone cells. Table III shows comparative disaccharide compositions of adenoma and carcinoma HS species which are different than a composition for bone-derived HS. Bone-specific HS is comprised of chains containing at least three, and up to eight distinct, highly sulphated, ligand-binding domains. Each domain is distinct in its disaccharide sequence, and is likely to bind a distinct extracellular ligand. A combination of ligands that these chains can bind is likely to assist in determining bone cell phenotype. Accordingly, the isolated HS of the present invention is clearly distinct from this previously characterised HS. The relative proportions of the six (6) major sulphated disaccharide groups in the bone HS chains are markedly different from any other published analysis, indicating that its bioactive domains are novel.

Prior to the present invention, HS had been prepared from non-bone tissues or bone-derived preparations were rather crude and comprised HS proteoglycan, i.e. not HS in isolation, but HS attached to a core protein, as described for example in Paine-Saunders et al, 2000, Dev Biol. 225 179 and McQuillan et al, 1991, Biochem 277 199, incorporated herein by reference. The vast majority of studies of glycosaminoglycan preparations from bone have related to chondroitin sulphate (which is a sugar that maintains joint fluid) and hyaluronan, which is exploited as an all-purpose “gel” capable of retaining and then releasing active growth factors.

The present invention relates to isolated HS that has been highly purified using SAX-HPLC after a combination of standard anionic exchange and gel filtration chromatography. As indicated above, the HS of the present invention is furthermore preferably obtained from isolated bone cells that are growing and differentiating.

HS controls activity of those growth factors that are absolutely crucial for tissue engineering applications currently being formulated as the “next wave” of biomedical therapy. Controlling the bioactivity of growth factors enables a fine control of tissue response parameters, e.g. bone repair. For example, HS regulates the bioactivities of the FGFs, PDGFs, TGF-betas, activins, the BMPs, HGFs, the pleiotropins, many cytokines and most of the effects of the adhesive components of the extracellular matrix. This has immense biological significance because this large variety of extremely potent, skeletally-active peptides (such as those listed above) is dependent on these compounds.

It will be appreciated that the HS of the invention may be used to stimulate tissue repair, both of hard and soft tissue. In a preferred embodiment, the invention is used to stimulate hard tissue repair, for example, repair of damaged bone. To this end, HS may be applied to implants, prosthesis and bioscaffolds to accelerate new bone formation at a desired location. It will be appreciated that heparan sulphates, unlike proteins, are particularly robust and have a much better ability to withstand the solvents required for artificial bioscaffolds and application to implants.

Coating an implant with HS of the invention may assist with anchoring or securing the implant to bone of a patient. Impregnating or coating a bioscaffold with HS may improve bone repair by stimulating bone cell growth and differentiation at a sight where a bone fragment is missing. Such use may enable a patient's own bone cells to repair a damaged area with need of a permanent artificial support matrix such as a hydroxyapatite-strengthened ceramic or plastic.

In addition to coating a biomaterial, for example an implant or bioscaffold, with HS one or more biologically active molecules may be absorbed over a coating of HS. For example, HS may be absorbed onto a biomaterial either via its anchoring core protein or after being derivatised on its reducing end. One or more biologically active molecules, for example, BMP2, BMP4, OP-1, FGF1, FGF2, TGF-β1, TGF-β2, TGF-β3, collagen 1, laminin 1-6, fibronectin or vitronectin may be absorbed over the absorbed HS at their respective active site. In addition to the above bioactive molecules, one or more bisphosphonates may be absorbed onto a biomaterial along with the HS. Examples of useful bisphosphonates may include etidronate, clodronate, alendronate, pamidronate, risedronate and zoledronate.

Implants and bioscaffolds coated or impregnated with HS of the invention may be useful in both human medical and veterinary purposes. It will be appreciated that the present invention may improve the quality of life of a patient or potentially extend the life an animal, for example a valuable race horse for use in breeding. The present invention may also be used for repair of damage to a dental structure.

HS of the invention may also be useful for determining and isolating a binding partner of a particular binding domain of HS. As an illustrative example, such a binding partner may be identified using affinity chromatography, where either a ligand or the HS is derivatised in turn to the chromatographic substrate. Another example of identifying a binding partner is plasmon resonance, where the HS may be immobilized on an aminosilane plate (for instance through the use of biotin) and the ligands are left soluble. Methods for isolating binding partners is described in Rahmone et al., 1998, Jour Biol Chem 273 7303, incorporated herein by reference. A biological function of an identified binding partner may be determined to ascertain if the molecule has biologically activity. In some embodiments, the molecule is capable of stimulating bone or bone cell growth and/or differentiation. The candidate molecule may be any natural or synthetic molecule.

### Increase Proliferation and Differentiation in MC3T3-E1 Cells

The MC3T3-E1 cell line has proven an important model system for studying the progression of bone development. It is able to reproduce all of the most important stages of bone development in a tissue culture environment. Despite this, most studies that have used this system have not exploited its full potential. For example, most studies have used confluent cells, usually after 3 or 4 days in culture, to assess a specific attribute, but do not continue with examination through subsequent developmental stages. The inventors assess herein MC3T3-E1 cells across all stages of growth.

Unlike previous investigations using this cell line, the inventors have surprisingly found that MC3T3-E1 cells are in fact density-dependent. This finding challenges previous studies according to which this cell line were density independent (Quarles, L. D., Yohay, D. A., Lever, L. W., Caton, R., and Wenstrup, R. J., 1992, J. Bone Min. Res. 7 (6) 683). Earlier studies claiming that MC3T3-E1 cells are density-independent were based on studies that ceased after only 15 days in culture. The inventors, however, observed that cells sloughed off a tissue culture plate upon reaching 100% confluence (whereupon a majority of cells died), usually after 17-18 days in culture.

Moreover, when MC3T3-E1 cells began to slough off the tissue culture plate surface, new underlying cell populations could be identified. This phenomenon raises some important developmental questions: for example, do these cells possess a potential to de-differentiate and divide or do some cells remain immature pre-osteoblasts that are a source of a continuous supply of cells.

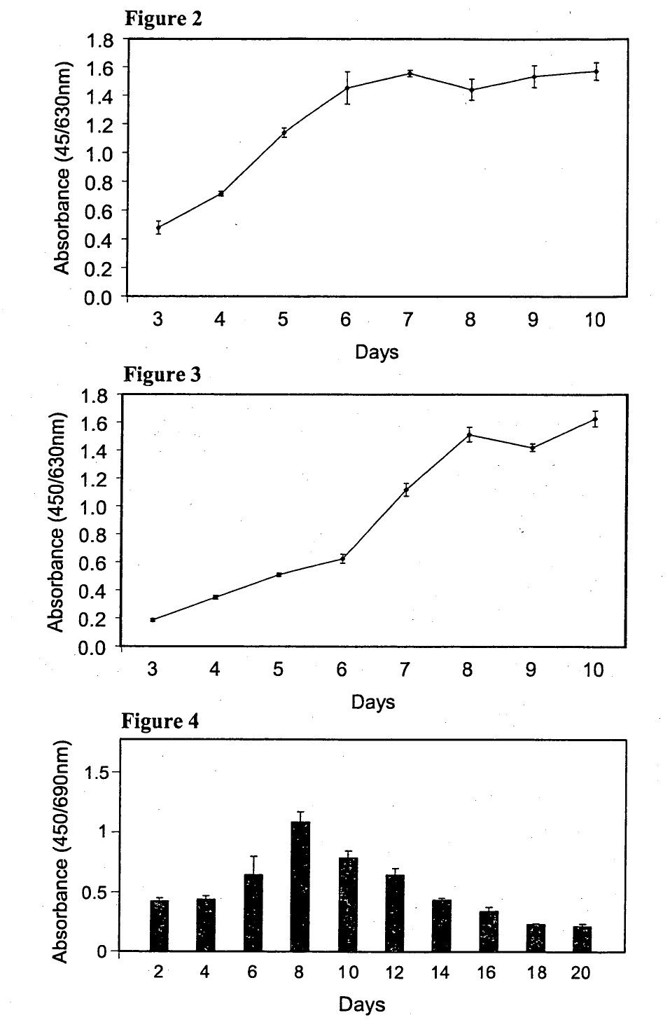

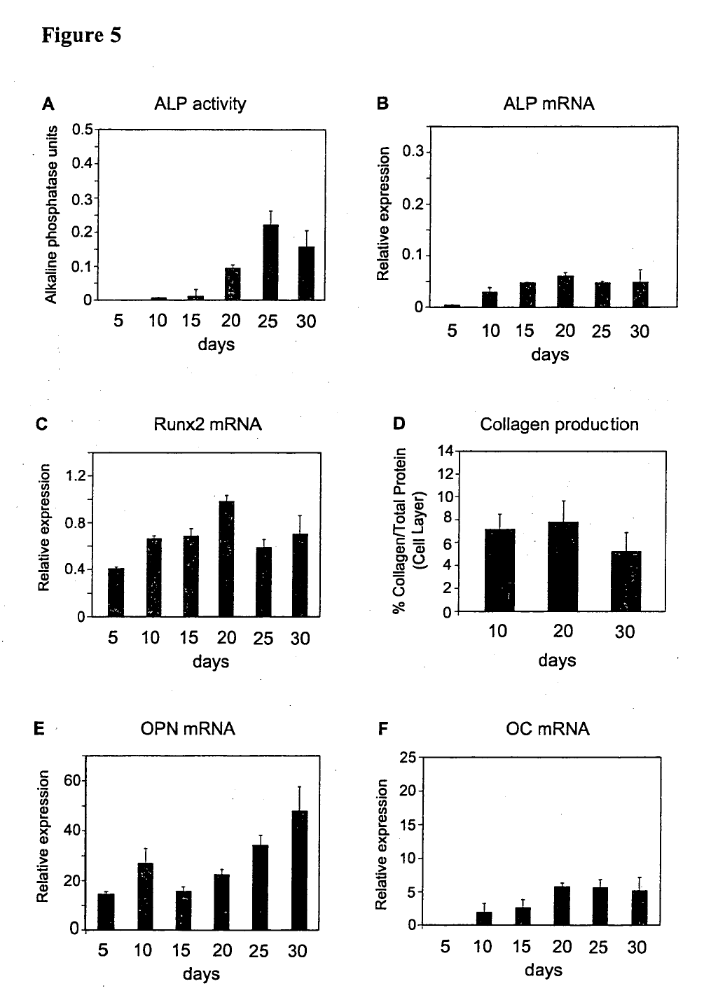

For MC3T3-E1 cells the inventors thus characterized an initial growth phase, lasting for about 6 to 8 days. After this period metabolic activity reaches saturation, while proliferation decreases accordingly (see FIGS. 2 to 4). After this initial growth phase cells start to differentiate (see FIG. 5). Cells in the initial growth phase can thus be described by a low expression of the marker proteins ALP, Runx2, OPN and OC (FIG. 5). The HSPG expression pattern of MC3T3-E1 cells does not reveal significant changes in the respective HSPG core proteins during the period where proliferation decreases and differentiation is initiated.

The inventors have found that expression of all four FGF receptors (FGFRs) is upregulated with increasing time in culture, independent of either phenotype or physical loading status. Once upregulated, receptor expression remained relatively constant, and no pattern could be discerned that linked overall FGFR configuration to a specific phenotype. From these observations it is plausible that these receptors are purely present in a constitutive manner.

However, although all four FGFR isotypes are present, they might not signal. FGFRs remain inactive in the membrane until dimerisation and subsequent transphosphorylation occurs after ligand binding. Both homomeric and heteromeric dimerisation can occur between FGFR isoforms (McKeehan and Kan, 1998, Prog Nucleic Acid Res Mol Biol. 59 135; Nurcombe et al, 2000, J Biol Chem. 275 (29) 30009; Ornitz and Itoh, 2001, Genome Biol. 2001 2 3005). Specific FGFRs can trigger proliferation and others differentiation, depending on such variables as ligand identity (Iseki et al., 1997, Development 124 3375), cross-linking heparan sulphate glycosaminoglycan moieties (Guimond and Turnbull, Curr Biol. 9 1343), and receptor occupation times (LaVallee et al. 1998, J Cell Biol. 141 1647). It is also possible that one half of the dimer-pair may be involved in both proliferation and differentiation, dimerising with different FGFRs (McKeehan and Kan Prog, 1998, Nucleic Acid Res Mol Biol. 59 135; Nurcombe et al, 2000, J Biol Chem. 2000, 275 30009).

The inventors have found that loading increases proliferation and differentiation of MC3T3-E1 cells, even though receptor expression remained constant. Not being bound by theory, this could be explained in two ways. The first is that loading is a mechanical stimulus, whereas receptors expression may be under the control of growth factor stimulation. Conceivably the FGFRs did not upregulate because there was no appropriate ligand. However, many cells possess large endogenous stores of FGFs, and autocrine release by cells may stimulate increases in receptor expression.

Ogata et al., 2000, J Cell Biochem. 76 529 examined effects of mechanical stimulation on tyrosine phosphorylation by shaking culture dishes comprising MC3T3-E1-osteoblast like cells; in particular they examined the ERK ½ signal transduction pathway. However, they did not explore FGF receptor profiles in these cells. They found an upregulation in ERK ½, Shc and egr-1 mRNA in response to fluid flow that is similar to effects seen with growth factor stimulation. Accordingly, effects of loading could be mediated through FGFRs.

Lisignoli et al, 2002, Biomaterials 23 1043, incorporated herein by reference, studied osteogenesis of large segmental radius defects in a rat model by implanting a biodegradable non-woven hyaluronic acid-based polymer scaffold (Hyaff 11) alone or in combination with bone marrow stromal cells (BMSCs). These cells had been previously grown in vitro in mineralising medium either supplemented with basic fibroblast growth factor (FGF-2) or unsupplemented. Healing of bone defects was evaluated at 40, 80, 160 and 200 days and the repair process investigated by radiographic, histomorphometric (assessment of new bone growth and lamellar bone) and histological analyses (toluidine blue and von Kossa staining). Mineralization of bone defects occurred in the presence of the Hyaff 11 scaffold alone or when combined with BMSCs grown with or without FGF-2, but each process had a different timing. In particular, FGF-2 significantly induced mineralization from day 40, whereas 160 days were necessary for direct evidence that a similar process was developing under the other two conditions tested (scaffold alone or with BMSCs). Radiographic score, new bone growth and lamellar bone percentage were highly correlated. According to these in vivo findings, the Hyaff 11 scaffold is an appropriate carrier vehicle for the repair of bone defects; additionally, it can significantly accelerate bone mineralization in combination with BMSCs and FGF-2.

The present invention thus also relates to a method of isolating HS from a tissue or cell, namely bone, a bone cell, a bone precursor cell and a stem cell. In one embodiment, the bone, bone cell, bone precursor cell or stem cell is obtained from a mammal. In some embodiments the mammal is a human, bovine, a pig or a rodent. In one embodiment, the bone cell, bone precursor cell or stem cell is cultured. The bone cell, bone precursor cell or stem cell may be isolated and cultured to remove other cell types. In other embodiments an available bone precursor cell line is used. Examples of suitable bone precursor cell lines include, but are not limited to, KS-4, UMR106, UMR201, MBA 15.4, 2T3, and MC3T3-E1.

The cultured cells, from which the HS is isolated, may be either in a logarithmic growth phase or contact inhibited. In a preferred embodiment, the cells are in a logarithmic growth phase.

In one embodiment, the method includes the steps of:
(i) fractionating culture media, membrane fraction and/or extracellular matrix fraction from bone, bone cells, bone precursor cells or stem cells by ion-exchange chromatography;
(ii) collecting an eluted fraction comprising glycosaminoglycans;
(iii) treating the collected fraction of step (ii) with neuraminidase;
(iv) treating the material of step (iii) with chondroitin ABC lyase;
(v) treating the material of step (iv) with pronase;
(vi) fractionating the material of step (v) by ion-exchange chromatography; and
(vii) collecting an eluted fraction comprising heparan sulphate.

Any ion-exchange chromatography using any separation media may be used for steps (i) and (vi). As an example, the ion-exchange chromatography of steps (i) and (vi) may be column chromatography and include the use of DEAE-Sephacel.

In one embodiment the collected fraction of step (ii) is desalted, freeze-dried and resuspended in a minimal volume.

Desalting may be performed by any means. Examples of respective means include, but are not limited to, ultrafiltration, dialysis, or gel filtration. As an illustrative example, desalting may be achieved by using a Centriflo Cone.

The neuraminidase of step (iii) and the chondroitin ABC lyase of step (iv) may be used at any concentration and any incubation conditions that are suitable of largely removing N-acetyl-neuraminic acid residues, largely degrading undesired polysaccharides, and at the same time leave HS largely, or, if desired, completely, unchanged. The respective undesired polysaccharides are mainly, but not only, chondroitin 4-sulphate, chondroitin 6-sulphate and dermatan sulphate.

The neuraminidase (sialidase), also called acetyl-neuraminyl hydrolase, of step (iii) may thus for instance be used at a concentration of 0.25 U/sample. The respective treatment may for instance last for four hours.

The chondroitin ABC lyase of step (iv) may for example be employed at a concentration of 0.25 U/sample and treatment may for instance last for four hours at 37° C. Additional chondroitin ABC lyase may be added for an overnight incubation.

It will be appreciated that an embodiment of the present invention provides isolated HS obtained from developing bone cells that are in active phase of growth and not already differentiated, a relatively pure form of HS with more complete characterization of sugars comprising the isolated HS and the HS of the invention comprise unique biological activity when compared with other HS preparations, including heparin.

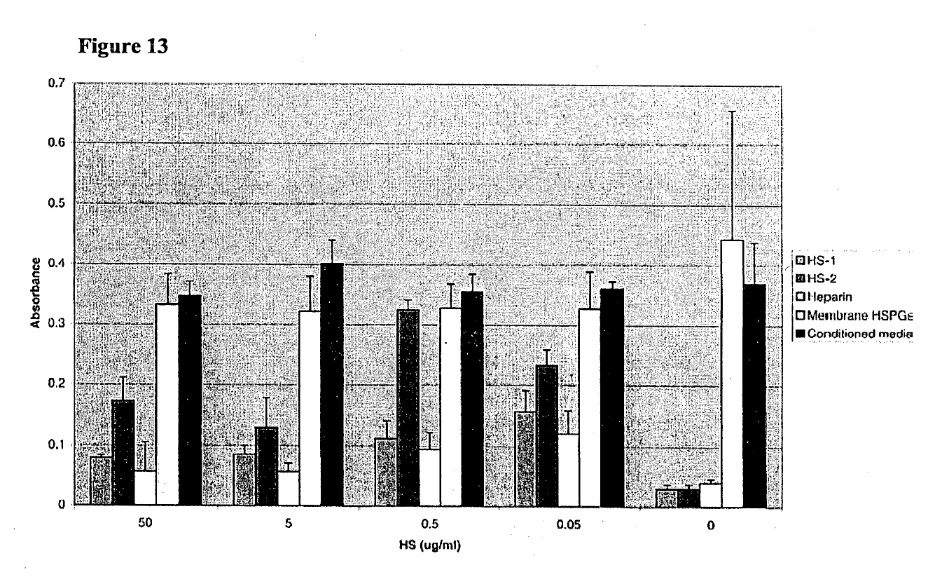

Such biological activity includes, for example, accelerating rates of growth of bone precursors by themselves, without supplementary growth factors. FGF-1 and FGF-2, which are known to stimulate bone cell growth (see above), induce a proliferative effect that is significantly weaker than the effect of 10% calf serum (see FIG. 12). HS isolated from brain cells also induces bone cell growth (FIG. 13). However, bone cell derived HS proved much more potent in this respect (FIG. 13). Accordingly HS, as isolated by the method of the present invention, is much more specific to growing bone cells.

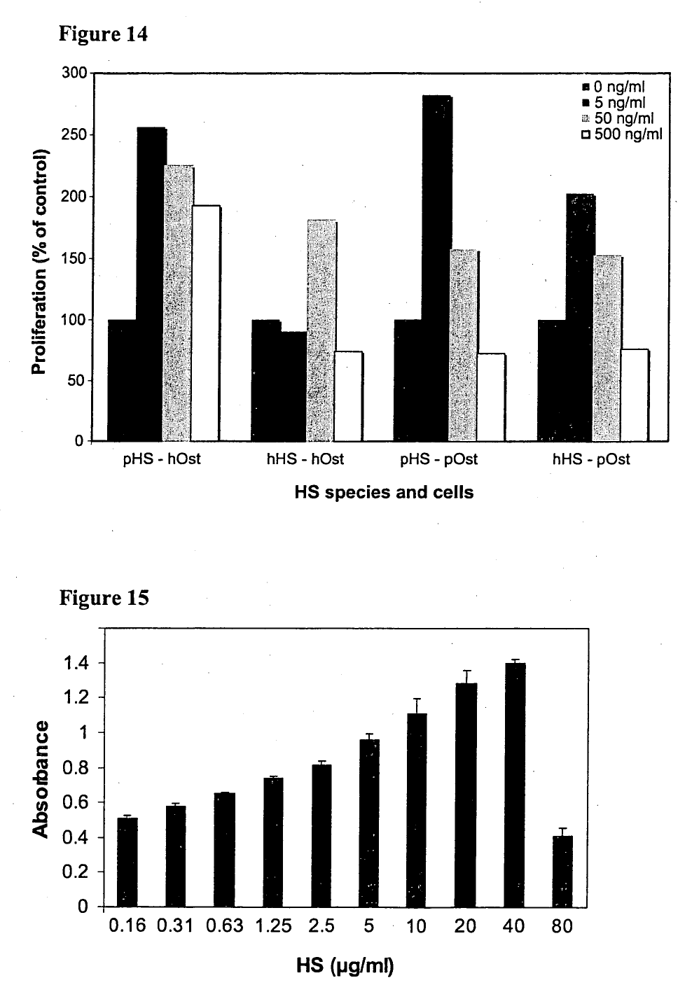

This stimulatory effect on bone growth was found to occur regardless of the source of the HS (see FIG. 14). Thus, the source of the HS may be selected independently from the species, in which it is desired to be used. The stimulatory effect on bone growth is further dose-dependant. The person skilled in the art will be aware of the fact that an optimal dose generally exists that may easily be determined in a standard experimental setup.

Thus, the HS may be part of a pharmaceutical composition. Such a composition may furthermore contain a carrier or diluent. Any carrier or diluent may be employed that does not obviate the biological activity of HS for which it is intended to be used. If desired, a carrier or diluent may be chosen that does not affect the biological activity of HS at all. Furthermore, the pharmaceutical composition may allow for a release of HS over any desired one or more time intervals. Thus, it may for example release the HS instantaneously or at one or more certain time points, over a period of minutes, over a period of hours or over a period of days.

A respective pharmaceutical composition may furthermore include biologically active molecules that are capable of stimulating bone or bone cell growth. Examples of such molecules include, but are not limited to, BMP2, BMP4, OP-1, FGF1, FGF2, TGF-β1, TGF-β2, TGF-β3, Collagen 1, laminin 1-6, fibronectin and vitronectin. The pharmaceutical composition may also include one or more bis-phosphonates. Examples of suitable bis-phosphonates include, but are not limited to, etidronate, clodronate, alendronate, pamidronate, risedronate and zoledronate.

A respective pharmaceutical composition may for example be used in the manufacture of a medicament for treating an animal in need of tissue repair.

The isolated HS (as described above and illustrated below) may furthermore be comprised in a surgical implant, prosthesis or bioscaffold. Any part of the surgical implant, prosthesis or bioscaffold may contain or consist of HS. As an example, a part of a respective implant, prosthesis or bioscaffold may be coated or impregnated with HS. Other components, which such a surgical implant, prosthesis or bioscaffold may comprise, include, but are not limited to, BMP2, BMP4, OP-1, FGF1, FGF2, TGF-β1, TGF-β2, TGF-β3, Collagen 1, laminin 1-6, fibronectin and vitronectin. As an illustrative example, the surgical implant, prosthesis or bioscaffold may also be coated or impregnated with such components. Examples of further components that a surgical implant, prosthesis or bioscaffold may comprise, include, but are not limited to, etidronate, clodronate, alendronate, pamidronate, risedronate and zoledronate. Using the above illustrative example, the surgical implant, prosthesis or bioscaffold may also be coated or impregnated with these latter components. Yet a further example of a component, which such a surgical implant, prosthesis or bioscaffold may comprise, is a polymer that incorporates hydroxyapatite or hyaluronic acid.

As an example, the surgical implant, prosthetic or bioscaffold may be used with hard tissue such as for instance bone. As another example, the surgical implant, prosthesis or bioscaffold may be used for the repair of dental damage.

In yet another aspect, the present invention relates to a method of treating an animal in need of tissue repair comprising a step of administering a pharmaceutical composition as described above. In some embodiments the animal is a mammal. Examples of a mammal that may be treated by the method of the invention include, but are not limited to, a human, bovine, a pig, or a rodent. Examples of a rodent that may be treated include, but are not limited to, a mouse, a rat or a guinea pig.

The tissue to be repaired in both afore mentioned aspects relating to an animal in need of tissue repair may be any tissue, such as for example soft or hard tissue. In some embodiments the tissue to be repaired is thus hard tissue. An example of suitable hard tissue is bone.

In one embodiment a respective repair of the hard tissue comprises a step of administering the pharmaceutical composition by coating or impregnating a surgical implant, prosthesis or bioscaffold as described above before implantation.

Any animal may be treated by this method of the invention. In some embodiments the animal is a mammal. Examples of mammals that may be treated by this method include, but are not limited to a human, bovine, a pig or a rodent. It may thus for example be obtained from a human.

The isolated heparan sulphate (see above) may furthermore be used for stimulating the regeneration of tissue. It may furthermore be used in the manufacture of a medicament for stimulating the regeneration of tissue. In this regard, the present invention also relates to a process of stimulating regeneration of tissue. This process includes a step of applying the HS, isolated as described above, to an area of the body of an animal in need of tissue regeneration. The HS may be applied over any desired one or more time intervals. Thus, it may for example be applied at one or more selected time points, over a period of minutes, over a period of hours or over a period of days.

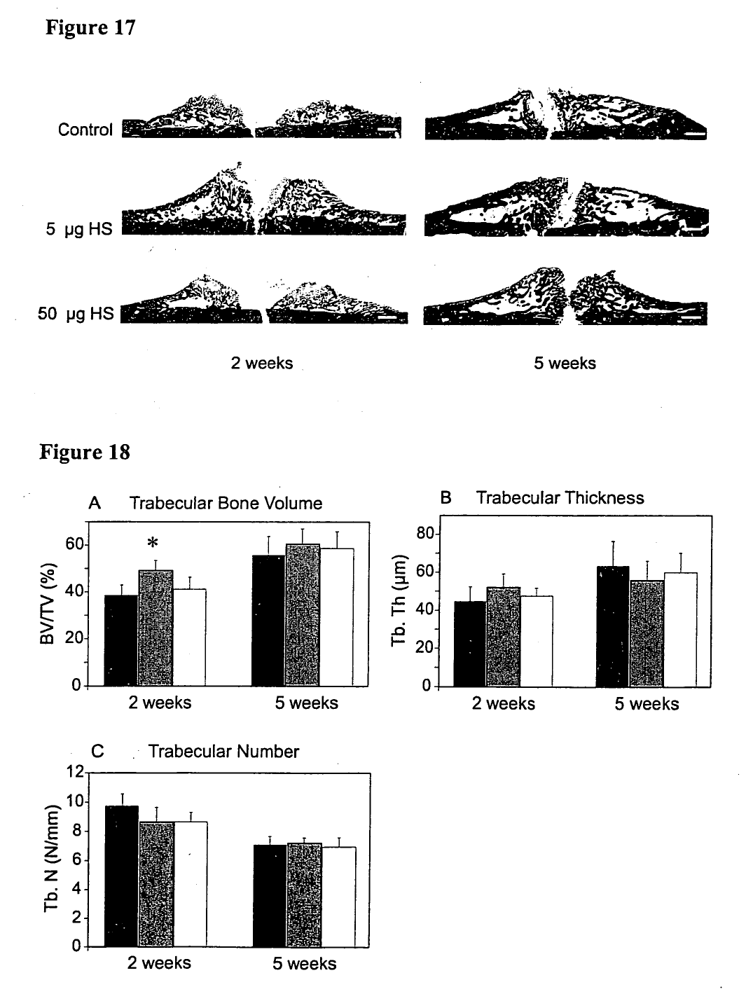

As an example, in embodiments where the respective tissue is bone, its regeneration is in one aspect due to the fact that the HS of the invention accelerates the growth of bone cells. In this case the need of repair may for instance relate to a fracture. Due to the accelerated growth of bone cells, such a fracture heals faster, although the healed bone will not be distinguishable from a bone healed without a treatment with HS (see FIGS. 17 to 19 and Table IV). Cartilage production as well as the number of osteoclasts remain unaffected by HS (see FIGS. 20 and 21). In another aspect the regeneration is due to the fact that the HS of the invention stimulates differentiation of a cell into a bone or bone-like cell. Typically, a respective cell is a precursor cell.

In this regard, the invention also relates to the use of HS, isolated as described above, for stimulating differentiation of a cell into a bone or bone-like cell. This use of HS may be conducted over any desired one or more time intervals. Thus, HS may for example be applied to a cell at one or more selected time points. As another example, HS may be applied to a cell continuously, for instance by means of a continuous release from a provided source, e.g. from a depot or by means of an infusion. Such a continuous application may last for any desired period of minutes, for example over a period of minutes, over a period of hours or over a period of days.

In some embodiments, such a cell is a stem cell. A non-limiting example of a stem cell is an embryonic stem cell. The use of isolated HS for the stimulation of cell differentiation may furthermore include the use of one or more biologically active molecules, which are capable of stimulating bone or bone cell growth and/or differentiation on the cells in addition to the heparan sulphate. Examples of suitable biologically active molecules include, but are not limited to, BMP2, BMP4, OP-1, FGF1, FGF2, TGF-β1, TGF-β2, TGF-β3, Collagen 1, laminin 1-6, fibronectin and vitronectin. The use of isolated HS for the stimulation of cell differentiation may also include the use of one or more bis-phosphonates. Examples of suitable bis-phosphonates include, but are not limited to, etidronate, clodronate, alendronate, pamidronate, risedronate and zoledronate.

In another aspect, the present invention relates to a method for identifying a biologically active molecule. The method includes the step of determining whether one or more candidate molecule(s) bind(s) to heparan sulphate, isolated as described above. In some embodiments this method further includes a step of determining a biological function of a respective molecule. The biologically active molecule is in some embodiments capable of stimulating bone or bone cell growth and/or differentiation. Examples of suitable biologically active molecules include, but are not limited to, a natural molecule, a synthetic molecule, an extract from a plant, an extract from animal, an extract from a tissue, an extract from a cell, a product from a recombinatorial library, a product from a cDNA library, a product from an expression library, a drug, a low molecular weight compound, a carbohydrate, and a protein. An illustrative example of a suitable protein is a growth factor.

### Definitions

Unless defined otherwise, all technical and scientific terms used herein have a meaning as commonly understood by those of ordinary skill in the art to which the invention belongs. Although any method and material similar or equivalent to those described herein can be used in the practice or testing of the present invention, preferred methods and materials are described. For the purpose of the present invention, the following terms are defined below.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form. For example, isolated HS may include extracts and purified HS obtained from bone MC3T3-E1 cells.

By “protein” is also meant “polypeptide”, either term referring to an amino acid polymer, comprising natural and/or non-natural amino acids as are well understood in the art. For example, HS may be coupled to a core protein. “Protein” may refer to a peptide, polypeptide, or fragments thereof.

By “heparan sulphate (HS)” is meant chains that are initially synthesised in the Golgi apparatus as polysaccharides consisting of tandem repeats of D-glucuronic acid (GlcA) and N-acetyl-D-glucosamine (GlcNAc). The nascent polysaccharides are subsequently modified in a series of steps: N-deacetylation/Nsulphation of GlcNAc, C5 epimerisation of GlcA to iduronic acid (IdoA), O-sulphation at C2 of IdoA and GlcA, O-sulphation at C6 of N-sulphoglucosamine (GlcNS) and occasional O-sulphation at C3 of GlcNS. N-deacetylation/N-sulphation, 2-O-, 6-O- and 3-O-sulphation of HS are mediated by the specific action of HS N-deacetylase/N-sulfotransferase (HSNDST), HS 2-O-sulfotransferase (HS2ST), HS 6-O-sulfotransferase (HS6ST) and HS 3-O-sulfotransferase, respectively. At each of the modification steps, only a fraction of the potential substrates are modified, resulting in considerable sequence diversity. This structural complexity of HS has made it difficult to determine its sequence and to understand the relationship between HS structure and function.

A “pharmaceutical composition” includes a composition comprising HS as an active ingredient. Suitably, the pharmaceutical composition comprises a pharmaceutically-acceptable carrier. By “pharmaceutically-acceptable carrier, diluent or excipient” is meant a solid or liquid filler, diluent or encapsulating substance that may be safely used or administration. Depending upon the particular route of administration, a variety of carriers, well known in the art may be used. These carriers may be selected from a group including sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Any suitable route of administration may be employed for providing a patient with the pharmaceutical composition of the invention. For example, coating or impregnating a surgical implant, prosthesis or bioscaffold. The invention may also be useful as a topical application for promoting wound healing of skin or other soft tissue. The present invention may be used medically as a pharmaceutical composition in a similar manner as described for oligosaccharides in WO 93/19096, incorporated herein by reference.

Dosage forms include suspensions, solutions, syrups, aerosols, gels, powders and the like. These dosage forms may also include implanting devices capable of controlled drug release designed specifically for this purpose or other forms of implants modified to act additionally in this fashion. The controlled release may be affected by using polymer matrices, liposomes and/or microspheres.

Pharmaceutical compositions of the present invention suitable for administration may be presented as discrete units such as vials, capsules, sachets or tablets each comprising a pre-determined amount of HS of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy, but all methods include the step of bringing into association HS of the invention as described above with a carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

In order that the invention may be readily understood and put into practical effect, particular embodiments will now be described by way of the following non-limiting examples.

### Materials

Trypsin was supplied by Calbiochem and DNase from Boehringer Mannheim. D-[6-³H]Glucosamine (sp. 21 Ci/mmol) was obtained from Amersham Life Science. Heparitinases I (EC 4.2.2.8), II (no EC number assigned) and III (EC 4.2.2.7) and chondroitin ABC lyase (EC 4.2.2.4) were obtained from Seikagaku Kogyo Co., Tokyo, Japan. Heparitinase IV was from Sigma (Sydney, Australia). Cell-culture media was supplied by Gibco. Bio-Gel P-2 and P-10 and the Trans-blot tank were from Bio-Rad Laboratories. CL-6B gel, DEAE-Sephacel, columns, peristaltic pumps, fraction collectors, and tubing were from Pharmacia Biotech Inc. (Sydney, Australia). ProPac PA1 analytical columns for the HPLC were from Dionex (Surrey, United Kingdom). Centriflo CF25 Membrane Cones were supplied by Amicon (Sydney, Australia). Scintillant (Ultima Gold) was from Packard (Melbourne, Australia) as were the scintillation vials. Biotrace RP nylon membrane was supplied by Gelman Sciences. En3Hance spray surface autoradiography enhancer was obtained from NEN Research Products, DuPont (U.K.) Ltd. Autoradiography cassettes were supplied by Genetic Research Ltd. X-Omat AR X-ray film and development chemicals were supplied by Kodak.

### EXAMPLES

### Example 1

### Cell Culture and Radiolabelling

Bone precursor MC3T3 cells were grown in 250 ml tissue culture flasks in 5% FCS/DMEM in a 10% CO₂/air-humidified incubator. When isolating logarithmic growth HS, radiolabel was added 24 h post-passaging and the cells allowed to grow unhindered for 3 days. To isolate HS from contact-inhibited cells, media on the cells was changed to 0.5% FCS/DMEM post-confluence and radiolabelled (20 μCi/ml) 24 h after the media was changed. Cells were maintained at confluence for 3 days and then the media collected and frozen at −20° C. until required. Cell membranes were prepared in lysis buffer (1% Triton X100, 150 mM NaCl, 10 mM Tris pH 7.4, 2 mM EDTA, 0.5% NP 40, 0.1% SDS containing the protease inhibitors 1 mM sodium orthovanadate, 10 μg/ml leupeptin, 1 μg /ml aprotinin and 1 mM PMSF). The cellular ECM was collected with lysis buffer plus 6 M Urea.

### Example 2

### Determination of Metabolic Activity Using WST-1

Unless otherwise indicated, MC3T3-E1 cells were plated at 5000 cells/cm² into wells of a 96 well plate in triplicate, allocating 3 wells to each time point, and grown in osteogenic media for 3-10 days. The Cell Proliferation Reagent WST-1 (Roche Diagnostics, Singapore) was added to triplicate wells at each time point, diluted 1:10 into the media. The reaction was catalysed by the conversion of WST-1, a tetrazolium salt, into formazon by mitochondrial dehydrogenase, which directly correlates to the number of metabolically-active cells in the culture. The reaction is incubated for 37° C. for 30 min, liberating a red colour, and read at 450 nm with a reference wavelength of 630 nm on a Victor3™ Multilevel Plate Reader (Perkin Elmer, Boston, Mass., USA). A blank well containing only media was used for background correction due to discolouration by the media.

As the assay can be performed and read under sterile conditions, cells for each time point can be plated in the same 96 well plate, thus, limiting differences due to plating, culture conditions and plastic. The conversion of WST-1 to formazan directly correlates to the number of metabolically-active cells in the culture. Metabolic activity increased up to about day 7 or 8 (FIG. 2, FIG. 3), after which time a plateau level was maintained, coinciding with confluence.

### Example 3

### Determination of Cell Proliferation Using BrdU

Cell proliferation was analysed with a Cell Proliferation ELISA colorimetric kit (Roche, Switzerland). MC3T3-E1 cells were incubated with 10 μM BrdU for 2 h at 37° C., denatured, fixed and incubated with anti-BrdU-POD for 90 min at RTP according to the manufacturer's instructions. The reaction was catalysed by the addition of a tetramethylbenzidine substrate solution and terminated after 15 min with 1 M H₂SO₄. The absorbance was read at 450 nm (with a reference of 690 nm) using a Bio-Rad® Benchmark™ Microplate Reader (Bio-Rad, CA, USA) and corrected using blank and background controls.

### Example 4

### Analysis of the Differentiation Status by Determining the Expression of Marker Proteins

Total protein and RNA were extracted from the cells and used for ALP-ELISA and real time PCR respectively.

For real time PCR, total RNA was isolated using the RNA Isolation Nucleospin® RNA II kit (Machery-Nagle, PA, USA) according to the manufacturer's instructions. RNA concentration was determined using a GeneQuant™ Pro RNA/DNA calculator (Amersham Biosciences) and the quality confirmed by RNA gel electrophoresis. RNA (1 μg) was reverse transcribed using Superscript™ II and Oligo dT12-18 Primer (Invitrogen, Singapore) according to the manufacturer's instructions. Oligonucleotides were designed using Primer Express® software, V2.0 (Chicago, Ill., USA) and synthesized by Research Biolabs (Singapore). The specific sequences are outlined below. Primer specificity was verified using the BLAST resource on the National Centre for Biotechnology Information (NCBI) website (http://www.ncbi.nlm.nih.gov/BLAST/). The PCR products of these primers were first tested using conventional PCR, and the products were sequenced by the IMCB Sequencing Facility (Singapore). Real Time quantitative PCR was performed on an ABI PRISM®7700 Sequence Detection System (Applied Biosystems, Foster City, Calif.) using SYBR® Green PCR Master Mix (Applied Biosystems, Foster City, Calif.) in triplicate wells. The reaction cycle consisted of a first stage for 10 min at 95° C. followed by 45 cycles of combined annealing and extension for 15 sec at 95° C. and for 1 min at 60° C. Primer concentration and efficiency were also determined using the same cycling conditions prior to conducting the assays. Results are expressed as a relative expression of hypoxanthine guanine phosphoribosyl transferase (HPRT) calculated using delta CT values.

A conversion of pNPP by ALP was detectable from day 10 onward (FIG. 5A), with maximal activity observed at days 25 and 30. In addition to ALP activity, ALP mRNA transcripts (FIG. 5B) were measured using real time PCR. ALP mRNA was detectable at all time points with a significant expression from day 10 onward. Runx2 mRNA expression (FIG. 5C) was detectable at all time points with an initial increase between days 5 and 10. Collagen synthesis (FIG. 5D) was measured at days 10, 20 and 30 using ³H-proline incorporation into collagenase-digestible proteins within the cell monolayer. OPN mRNA expression (FIG. 5E) was increasing after day 15. OC mRNA expression was detectable from day 10 onward, increasing until day 20.

The following mouse oligonucleotides were used:

Acc. No=the Genbank accession number for the mRNA sequences. F=forward primer, R=reverse primer, size=the PCR product size.

| | | | | |
|---|---|---|---|---|
| | | SEQ | | |
| | | ID | | |
| Transcript | Acc. No | No | Sequence (5′-3′) | Size |

| | | | | | |
|---|---|---|---|---|---|
| ALP | X13409 | F | 1 | GAT AAC GAG ATG CCA CCA GAG G | 140 |
| | | R | 2 | TCC ACG TCG GTT CTG TTC TTC | |
| OP | BC057858 | F | 3 | CCA GGT TTC TGA TGA ACA GTA TCC | 163 |
| | | R | 4 | ACT TGA CTC ATG GCT GCC CTT T | |
| Runx2 | NM_009820 | F | 5 | ACA AAC AAC CAC AGA ACC ACA AGT | 111 |
| | | R | 6 | GTC TCG GTG GCT GGT AGT GA | |
| OC | U11542 | F | 7 | GAG GGC AAT AAG GTA GTG AAC AGA | 134 |
| | | R | 8 | AAG CCA TAC TGG TTT GAT AGC TCG | |

### Example 5

### Preparation of Intact Heparan Sulphate Chains

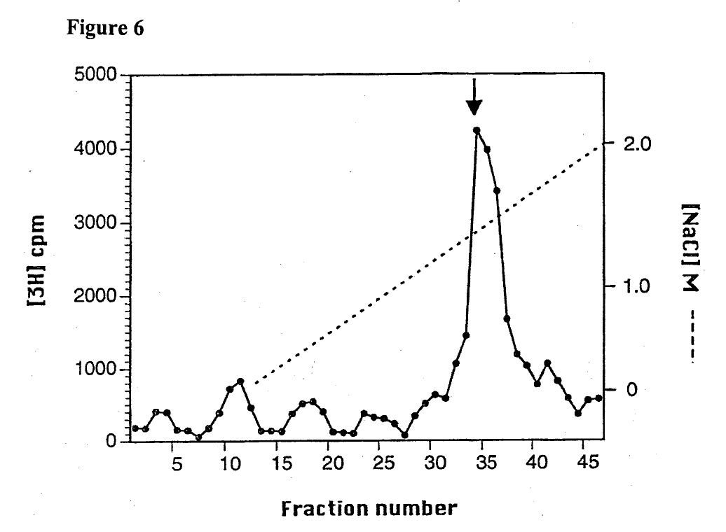

The cellular extracts were subjected to ion-exchange chromatography on a DEAE-Sephacel column equilibrated in 150 mM NaCl with phosphate buffered saline (PBS), pH 7.2. The media was manually loaded onto the column and eluted under gravity (FIG. 6). As shown, most of the radioactivity elutes in a single peak between 1.0 and 2.0 M NaCl. An arrow indicates bone derived HS material that was collected and used for further analysis. The column was washed and the bound material eluted with 2M NaCl in 50 mM PBS and 2 ml fractions collected.

Fractions comprising the 3H-glucosamine labelled GAGs were pooled, concentrated and desalted, freeze dried and resuspended in a minimal volume (100-500 μl) of neuraminidase buffer (25 mM Na-acetate pH 5.0). Samples were treated with neuraminidase (0.25 U/sample) for 4 h. Five volumes of 100 mM Tris-acetate (pH 8.0) were then added to the sample which was then digested with chondroitin ABC lyase (0.25 U/sample) for 4 h at 37° C. and further digested overnight with an equal amount of fresh enzyme.

Finally, the core protein and the lyases were digested away with Pronase (⅕ total volume of 10 mg/ml Pronase in 500 mM Tris-acetate, 50 mM calcium acetate, pH 8.0) at 37° C. for 24 h. The entire mixture was then diluted 1:10 with deionised water, passed through a 2 ml DEAE-Sephacel column, eluted as previously described and 1 ml fractions collected.

The sample was finally desalted on a 1×35 cm Bio-Gel P2 column, the Vₒ fraction collected and freeze dried. Samples were then eluted in a ˜200 μl of 500 mM NaOH/1M NaBH₄, incubated for 16 h at 4° C. and then neutralised to pH 7 with glacial acetic acid.

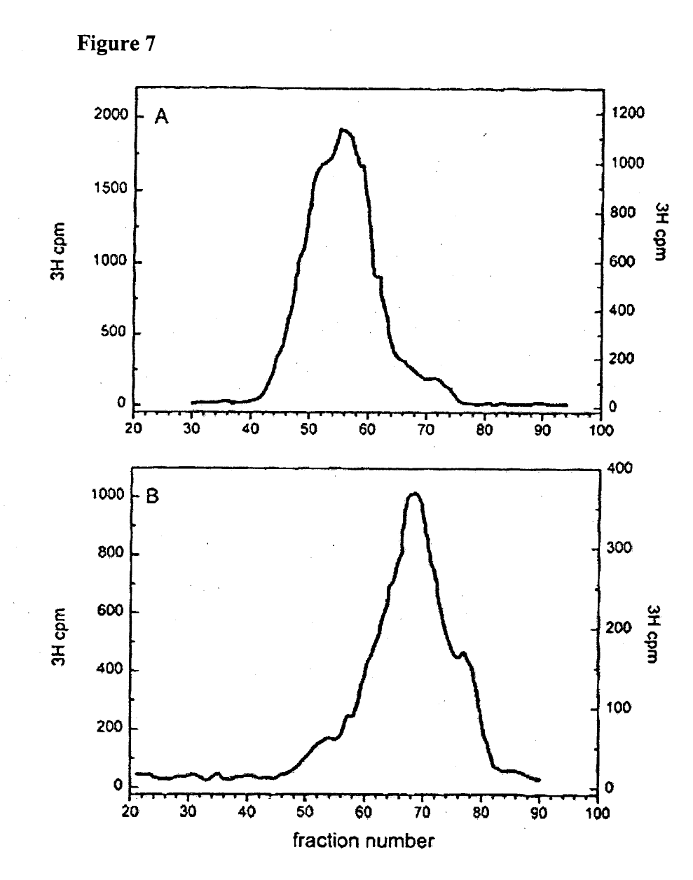

A small amount of saturated ammonium bicarbonate was added and samples run on a CL-6B column (1×120 cm) for size determination of the released HS chains (FIG. 7). Size of full length HS (A) and heparinase-resistant fragments (B) can be calculated from these graphs. Results are summarized in Table II.

### Example 6

### Nitrous Acid Treatment of HS Chains

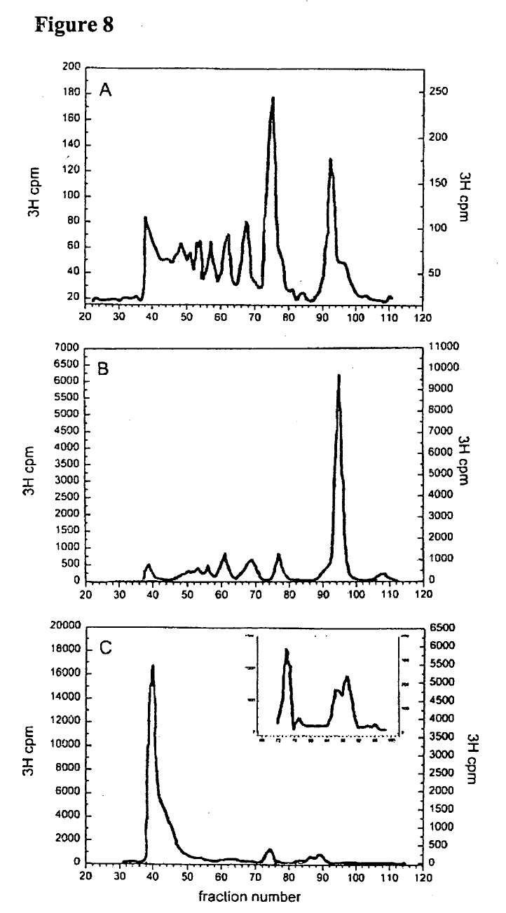

HS was chemically depolymerised by low pH-HNO₂ (pH<1.5). A small portion of the mixture was run on a Bio-Gel P10 column (1×200 cm) to obtain a profile of the fragments released by this treatment (shown in FIG. 8A and Table I). This profile was used to determine purity of the HS sample and to calculate a percentage of susceptible linkages (Table I). A large fraction of this sample was separated on a Bio-Gel P-2 column (1×120 cm) to isolate disaccharides and tetrasaccharides for strong anion exchange-high pressure liquid chromatography (SAX-HPLC).

### Example 7

### Lyase Depolymerisation of HSPGs

A profile of depolymerised products treated with heparitinase is shown in FIG. 8B. Susceptibility of each species to heparitinase was calculated from this profile and tabulated. Degree of polymerisation (dp) of each peak is represented by the number above that peak and was subsequently used in the calculations. Heparitinase (heparitinase I), heparitinase II and heparitinase IV were used at a concentration of 25 mU/ml in 100 mM-sodium acetate/0.2 mM-calcium acetate, pH 7.0.

FIG. 8C shows a profile of depolymerised products treated with heparinase. Inset shows fractions 64-115 of the heparinase scission profile with an expanded scale in order to reveal the proportions of low-Mr products. Non-resolved Vo peak was pooled, freeze-dried and resolved on a Sepharose CL-6B as above. Heparinase was used at a concentration of 50 mU/ml in the same buffer.

Samples respectively treated with heparitinase or heparinase were digested in the presence of 100 μg of carrier HS. Each sample was separately incubated at 37° C. for 16 h and then a second aliquot of enzyme added and incubated for a further 4 h. Sequential digests for recovery of disaccharides for SAX-HPLC analysis were performed at 37° C. as follows: heparinase for 2 h, heparitinase for 1 h, heparitinase II for 18 h, and finally an aliquot of each lyase and heparitinase IV for 6 h. Sample volumes were decreased to less than 100 μl by desiccation and run on a Bio-Gel P-2 column to isolate disaccharides. Results are shown in Table II.

### Example 8

### Gel Chromatography

Gel chromatography of intact chains or scission products was performed on Sepharose CL-6B (1×120 cm) columns in a running buffer of 0.5M NH₄HCO₃ as shown for example in FIG. 6. Samples were eluted at 4 ml/hr with 1 ml fractions collected. Estimates of the size of fragments resolved on Sepharose CL-6B were based on our published calibrations.

### Example 9

### SAX-HPLC Analysis of Disaccharides and Tetrasaccharides

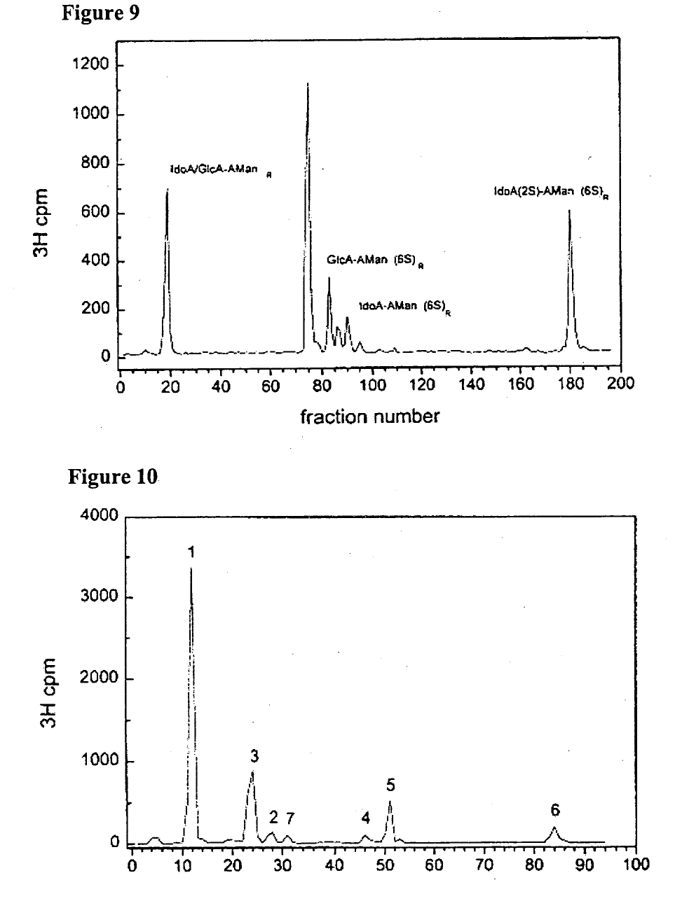

Disaccharide composition of the HS was analysed on strong anion exchange-high pressure liquid chromatography (SAX-HPLC) after either complete depolymerisation with a mixture of lyases as described above (FIG. 9; Table II) or HNO₂ treatment (FIG. 10; Table I). Disaccharides and/or tetrasaccharides were recovered by gel chromatography (Bio-Gel P-2 column) and fractions corresponding to disaccharides or tetrasaccharides were pooled, freeze-dried and stored at −20° C. before separation by SAX-HPLC.

Lyase-derived disaccharides were subjected to SAX-HPLC on a ProPac PA1 analytical column (4×250 mm) as follows. After equilibration in the mobile phase (double-distilled water adjusted to pH 3.5 with HCl) at 1 ml/min, samples were injected and disaccharides eluted with a linear gradient of NaCl from 0-1 M over 45 min in the same mobile phase. The eluant was collected in 0.5 ml fractions and the radioactivity measured by scintillation counting for comparison with lyase-derived disaccharides standards. In FIG. 9, each peak is labeled and a summary of proportions of each peak is provided in Table II.

Nitrous acid-derived tetrasaccharides were subjected to the same conditions (with smaller fractions collected) and compared to double labelled standard results which were supplied by Dr. Gordon Jayson (Christie Hospital, Manchester, UK). Alternatively, HNO₂-derived disaccharides were separated using two ProPac PA1 columns in the same mobile phase. A shallow, non-continuous gradient was used over the course of 97 min. From 0-51 min a gradient from 0-150 mM NaCl was employed and from 52-121 min a gradient of 150-500 mM NaCl was used.

Eluant was collected as described above and compared to standards. As shown in FIG. 10, elution peaks were labeled accordingly based on a comparison to authentic standards as described above. The relative amounts of each of peak has been calculated and summarised in Table I.

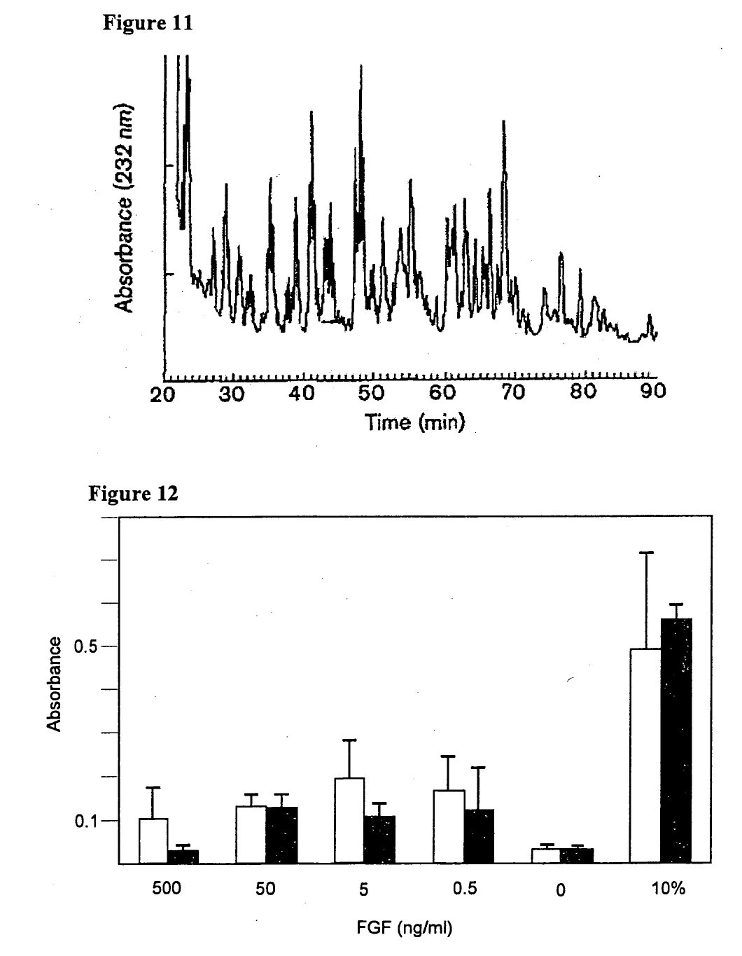

FIG. 11 shows a profile used to prepare an HS disaccharide total profile/library by high resolution SAX-HPLC. Following treatment with heparitinase, saccharide products were fractionated by size exclusion chromatography as described above to produce size-defined mixtures from dp4 to dp20 (4-20 monosaccharide units). A library of 32 structurally diverse decasaccharide fractions was then fingerprinted. Some are single peaks, others are tightly clustered groups of peaks representing isomers with slight structural variation.

### Example 10

### Effects of FGF-1 and FGF-2 on Proliferation of MC3T3-E1 Bone Cells

FIG. 12 is a graph showing MC3T3-E1 cell proliferation as monitored by BrdU incorporation in response to FGF-1 (black bars) and FGF-2 (white bars) respectively. Different concentrations of FGF-1 or FGF-2 as shown were respectively added to MC3T3-E1 cells and proliferation monitored. A positive control is 10% foetal calf serum.

FIG. 12 is a control experiment that shows that MC3T3-E1 cells are responsive to FGF-1 and FGF-2 when presented to them without HS supplementation, but that the addition of foetal calf serum greatly overwhelms (i.e. is much greater than) this response. The cells are responsive to the other factors in FCS that are not attributable to just FGFs.

### Example 11

### Comparison of Cell Proliferation by Bone-Derived HS and Other HS Sources

FIG. 13 is a graph illustrating effects of HS supplementation on proliferation of MC3T3-E1 bone cells. HS was prepared by DEAE ion-exchange chromatography and CL-6B filtration as described above. HS1 is a purified HS specific for the growth factor FGF-1 isolated from brain precursor cells; HS2 is a purified HS specific for the growth factor FGF-2 isolated from brain precursor cells; heparin is a non-bone derived, hypersulphated, clinically used HS (the so-called “gold standard”, in that it shows little or no specificity for ligands that are not involved in anti-thrombin III cascades) isolated from porcine mast cells; membrane HSPGs is bone HS purified from bone cell membranes (includes HS proteoglycans) and conditioned media is bone HS secreted into culture media away from bone membranes (two different HS bone cell derived compartments). Both HS bone cell derived compartments, i.e. membrane and excreted are shown having equipotent activity.

Cell proliferation was monitored by BrdU as described in example 3.

The concentration dependencies in FIG. 13 show a typical bell-shape for each HS. Generally, an optimal concentration of an effect on proliferation is observed, since inhibitory side effects occur at high HS concentrations. As an example, HS-2 shows its optimal stimulatory effect around a concentration of about 0.5 μg/ml in this case.

FIG. 13 further demonstrates that the HS secreted by bone cells is substantially more potent than the purified FGF-binding HS obtained from brain precursor cells. This strongly suggests that the bone cells require other HS-binding mitogens than just FGFs in order to grow at optimal rates. The “raw” bone HS fractions are binding an optimal ratio of tissue-specific factors.

### Example 12

### Comparison of Cell Proliferation by HS From a Different Species

FIG. 14 illustrates the effects of HS supplementation from a different species on proliferation of osteoblasts. HS was prepared by DEAE ion-exchange chromatography and CL-6B filtration as described above. Human HS (hHS) and porcine HS (pHS) was added to pig osteoblasts (pig HOst) and human osteoblasts (hOst) in all four combinations, as depicted in FIG. 14. The respective osteoblasts were isolated by standard procedures well known in the art. Proliferation was measured over a 24 h period as described above. 0.5, 5 and 50 ng/ml of the respective HS was added and the effect compared to a control (0 ng/ml). An increase of proliferation was observed in all cases. Again, an optimum can be observed for each combination. This optimum for porcine HS was in this case observed to be about 5 ng/ml, while about 50 ng/ml was found to reflect the respective optimum for human HS. Both optima were observed irrespective of the osteoblast source. Thus, no significant difference was observed between purified HS from another species and purified HS from the same species.

### Example 13

### Disaccharide Analysis

### Isolation of HS from Bone Tissue

Bone samples are removed from an animal, for example a rat, rabbit or cow. The bone sample is ground up at −20° C. in 150 mM NaCl with phosphate buffered saline (PBS), pH 7.2, first with mortar and pestle, then with a standard tissue homogenizer (10 passes), then with the ultraturrax. The homogenate is then gently removed and centrifuged (1000 rpm for 5 min) to remove any cell debris and stored at −20 ° C. until required. The media is subjected to ion-exchange chromatography on a DEAE-Sephacel column (3 ml) equilibrated in 150 mM NaCl with phosphate buffered saline (PBS), pH 7.2. The media is manually loaded onto the column and eluted under gravity. The column is washed with 10 column volumes of 250 mM NaCl in 50 mM PBS, pH 7.2. Bound material (primarily HS, CS and DS) is eluted with 1 M NaCl in 50 mM PBS and 2 ml fractions collected. Fractions comprising ³H-glucosamine labelled GAGs (primarily fractions 1-3) are pooled, concentrated and desalted on Amicon concentration cones as per manufacturers instructions, freeze-dried and resuspended in a minimal volume (100-500 ml) of neuraminidase buffer (25 mM Na-acetate pH 5.0).

Samples are treated with neuraminidase (0.25 U/sample) for 4 h. Five volumes of 100 mM Tris-acetate, pH 8.0 is added and chondroitin sulphate and dermatan sulphate digested by addition of chondroitin ABC lyase (0.25 U/sample) for 4 h at 37° C. and further digested overnight with fresh enzyme. Finally core proteins and all of the lyases will be digested with Pronase (⅕ total volume of 10 mg/ml Pronase in 500 mM Tris-acetate, 50 mM calcium acetate, pH 8.0) at 37° C. for 24 h.

The entire mixture is diluted to 1:10 with water, passed through a 2 ml DEAE-Sephacel column, eluted as previously described, and 1 ml fractions collected. The sample is finally desalted on a 1×35 cm Bio-Gel P2 column and the Vₒ fraction collected, freeze-dried and stored until needed.

### Heparan Sulphate Characterisation

To remove HS chains from the core protein, samples are incubated in 500 mM NaOH/1 M NaBH₄ for 16 h at 4° C. and neutralised to pH 7 with glacial acetic acid. Concentrated ammonium bicarbonate is added and after bubbling has stopped, samples are run on a CL-6B column (1×120 cm) for sizing of released HS chains. For HS depolymerisation reactions, heparitinase (heparitinase I), heparitinase II and heparitinase IV are used at a concentration of 25 mU/ml in 100 mM-sodium acetate/0.2 mM-calcium acetate, pH 7.0. Heparinase is used at a concentration of 50 mU/ml in the same buffer. Samples are digested in the presence of 100 mg non-labelled carrier HS (porcine mucosal HS). Each sample is separately incubated at 37° C. for 16 h and then a second aliquot of enzyme added and incubated for a further 4 h. For preparation of total disaccharides for SAX-HPLC analysis, sequential digests comprising 100 mg non-labelled HS is digested at 37° C. as follows: heparinase for 2 h followed by heparitinase for 1 h and then heparitinase II for 18 h, and finally an aliquot of each lyase and heparitinase IV for 6 h. Samples are dried down to less than 100 ml and run on a Bio-Gel P-2 column (1×120 cm) to desalt and remove all excess protein.

Gel chromatography of intact chains or scission products is performed on Sepharose CL-6B (1×120 cm), Bio-Gel P-2 (1×120 cm) and Bio-Gel P-10 (1×200 cm) columns. Running buffer for CL-6B and the Bio-Gel P-10 columns is 0.5 M NH₄HCO₃ and for Bio-Gel P-2 column is 0.25 M NH₄HCO₃. Samples are routinely eluted at 4 ml/h with 1 ml fractions collected. For preparative runs, radioactivity of a small aliquot of each fraction (1-10 ml) is monitored by liquid scintillation counting to ensure good separation and accurate isolation of fragments for further analysis. Estimates of the size of fragments resolved on Sepharose CL-6B is based on published calibrations.

### Disaccharide Analysis

Disaccharide composition of the HS is analysed on SAX-HPLC after either complete depolymerisation with a mixture of lyases or HNO₂ treatment. Disaccharides and/or tetrasaccharides are recovered by Bio-Gel P-2 chromatography and fractions corresponding to disaccharides or/and tetrasaccharides are pooled separately, freeze-dried and stored at −20° C. HNO₂-derived disaccharides are separated using 2 ProPac PA1 columns in series in the mobile phase (double-distilled water adjusted to pH 3.5 with HCl) at 1 ml/min. A shallow, non-continuous gradient is used over a course of 97 min. After a 1 min injection phase, a 50 min gradient from 0-150 mM NaCl is used followed by a 70 min gradient of 150-500 mM NaCl. The eluant is either collected (0.25 or 0.5 ml fractions) or monitored in-line using a radiomatic Flo-one/Beta A-200 detector (Canberra Packard, Pangbourne, United Kingdom) and compared to authentic standards.

Major peaks are labelled in FIGS. 9 and 10 and three minor disaccharide peaks eluted as follows: GlcA(2S)-AMannR between 43.75 and 44 min, GlcA-AMannR(3S) between 45.75 and 46.5 min and GlcA-AMannR(3,6S) between 104 and 106 min.

Lyase-derived disaccharides are subjected to SAX-HPLC on a ProPac PA1 analytical column (4×250 mm, Dionex Ltd.). After equilibration in the same mobile phase at 1 ml/min, samples are injected and disaccharides eluted with a linear gradient of sodium chloride from 0-1 M over 45 min. Fractions are collected and monitored for 3H-labelled disaccharide content. Nitrous acid-derived tetrasaccharides are subjected to the same SAX-HPLC conditions. Tetrasaccharides are compared to double labelled standard results which will be supplied by Dr. Gordon Jayson (Christie Hospital, Manchester, UK).

### Example 14

### Use of Heparan Sulphate of the Invention With Implant and Bioscaffold

Isolated HS is biotinylated (by incubation in 0.1 M MES buffer (pH. 5.5 with 50 mM biotin hydrazide and 10 mM N-ethyl-N′(dimethylaminopropyl)-carbodiimide) for 5-6 h at room temperature. The biotinylated HS is separated from excess reagent on a PD-10 column and virtually irreversibly immobilized to any streptavidin-coated surface. Such methods can be used to integrate HS into bioscaffolds of virtually any synthetic, biologically inert therapeutic material.

In one embodiment. HS is used in a relatively “raw” form (ie. not highly purified, or broken down into particular active, sulphated domains), so that the HS can interact with a correct proportion of tissue-specific growth and adhesive factors for which it is designed. For example, HS could be integrated it into scaffolds of hydroxyapatite or hyaluronic acid for wound/fracture repair. Alternatively, or in addition, HS may be purified from a HS mix (one-by-one) and HS specific for each factor that a tissue needs for growth/regeneration. HS is thereby acting as “bait” for essential factors that a growing/regenerating tissue requires.

### Example 15

### Analysis of the Dose Dependency of HS on Cell Proliferation of MC3T3-E1 Bone Cells

The in vivo doses of HS were determined using a cell proliferation enzyme-linked immunosorbent assay (ELISA) kit (Roche, Switzerland). Twenty-four hours prior to seeding, MC3T3-E1 cells were grown in starving media containing 50 mM NaClO₃ to disrupt the sulphation of endogenous HS. Cells were then seeded in starving media at a density of 1×10⁴ cells per well in a 96 well multi-titre plate, and incubated at 37° C. in 5% CO₂ for 1 h to allow for cell attachment. Following this, the media was replaced with serial dilutions of HS in starving media for 24 h, using serial dilutions of media containing 10% FCS as the assay control. Cells were then incubated with 10 μM BrdU for 2 h at 37° C., denatured, fixed and incubated with anti-BrdU-POD for 90 min at RTP according to the manufacturer's instructions. The reaction was catalysed by the addition of a tetramethylbenzidine substrate solution and terminated after 15 min with 1 M H₂SO₄. The absorbance was read at 450 nm (with a reference of 690 nm) using a Bio-Rad® Benchmark™ Microplate Reader (Bio-Rad, CA, USA) and corrected using blank and background controls. The assay was repeated three times and the 50% effective concentration value (ED50) was determined to be ˜5 μg/ml (FIG. 15).

### Example 16

### Comparison of HS Composition From Bone and Non-Bone Derived Sources

As previously described by Jayson et al, 1998, Jour. Biol. Chem 273 51, incorporated herein by reference, disaccharide composition is different for HS isolated from difference sources. Table III below shows comparative disaccharide compositions of the adenoma and carcinoma HS species. HS samples were degraded by combined heparinase I, II, and III digestion, and resulting disaccharides were analyzed by SAX-HPLC. The results represent the mean of values obtained from three determinations, with the S.E. values in all cases being 1.5%.

### Example 17

### Analysis of the the Acceleration of the Healing Process of a Bone Fracture by HS

### Surgical Procedure

Ninety 10-week-old male Wistar rats were anaesthetized using 75 mg/kg of ketamine and 10 mg/kg of xylazine by intraperitoneal injection. After sterile preparation, a 2 cm longitudinal incision was created along the lateral aspect of the thigh, the musculature carefully separated, and the dissection taken down until the femur could be adequately visualised. Periosteum was stripped from the bone, and a transverse osteotomy created in the femoral midshaft using a Stryker sagittal saw (Kalamazoo, Mich., USA). A Stryker TPS microdriver was then used to drill a 1.1 mm smooth K-wire down the intramedullary canal of the distal cut end of the femur and out at the knee, until it sat flush with the end of the bone. The fractured femur was then reduced and aligned, and the K-wire drilled retrograde in the medullary canal until it could be felt in the hip. The wire was then trimmed to reduce the likelihood of inflammation in the knee. The gel (100 μl) with or without HS was injected around the anterolateral aspect of the fracture site. The muscle, fascia and skin were then re-approximated and sutured, and the rat given 0.05 mg/kg Temgesic for pain relief immediately, as well as 12 hours post-operatively.

### Callus Size

Following euthanasia, both limbs were excised and freed from muscle. Using forceps, the intramedullary wires were removed and the callus size measured in the anterior-posterior (AP) and lateral planes using a digital Vernier Calliper (Sealey, UK). Bilateral femurs were then immersed in 4% PFA in 15 ml tubes. The obtained results showed that by 2 weeks, callus size in the AP plane was 23% larger in response to 5 μg HS when compared to the control and 50 μg HS groups (p<0.05, Table IV), with no difference between the groups detected in the lateral plane. At 5 weeks, no difference was detected in either the AP or lateral planes for the 3 groups. Thus the healing process was accelerated with HS, but reached the same endpoint as the control group.

### X-Ray and Quantitative Computerized Tomography

Radiographs of the right and left femurs were taken at a distance of 100 cm in the AP plane (see FIG. 16). Fracture healing was graded by 2 blinded orthopaedic surgeons. Peripheral quantitative computer tomography (pQCT) was then conducted using a Stratec XCT-960A pQCT scanner and analysis software (Stratec Medizintechnnik Gmbh, Germany). Nine, 1 mm slices were taken through the femoral mid-shaft, with the 5th slice through the original fracture site. To assess for a systemic effect of HS, two slices were taken 10 mm apart in the contralateral limb, corresponding to areas of cortical and cancellous bone.

### Trabecular Bone Formation

In order to determine whether the increase in callus size in the 5 μg HS group was due to increased trabecular bone formation, resin embedded sections were stained with 1% silver nitrate to measure the percentage of von Kossa-positive trabecular bone volume formation within the total callus volume (BV/TV). Using Bioquant analysis software, bone volume (BV), total volume (TV) and bone perimeter (BP) measurements were taken from the callus regions on each slide, with no less than 9 samples per group examined. These measurements were used to determine BV/TV, as well as trabecular thickness (Tb. Th; [(BV×2)/TV]) and trabecular number (Tb.N; [(BP×0.5)/TV×1000]). These measurements were averaged for each group, and the results presented as the mean±standard deviation.

Histomorphometric measurements showed a 19.6% increase in BV/TV with 5 μg HS compared to the control group (p<0.05, FIG. 18), suggesting that increased bone formation caused in the increase in callus size. In contrast, no difference was observed between the 50 μg HS and control groups. These differences are shown in FIG. 18, with a greater mineralized tissue and osteoid present at the bone/cartilage interface within the middle of the callus in the 5 μg HS group as compared to the 50 μg HS and control groups. The results also show that 5 μg HS increased Tb Th. by 16.5% as compared to control, whilst Tb N was equal amongst the 3 groups, suggesting that increased BV/TV may have been due to increased Tb Th. rather than Tb N. By 5 weeks, BV/TV, Tb Th. and Tb N measurements were equal across the 3 groups, indicating that these fractures were all at the same stage of healing.

### Safranin O Staining

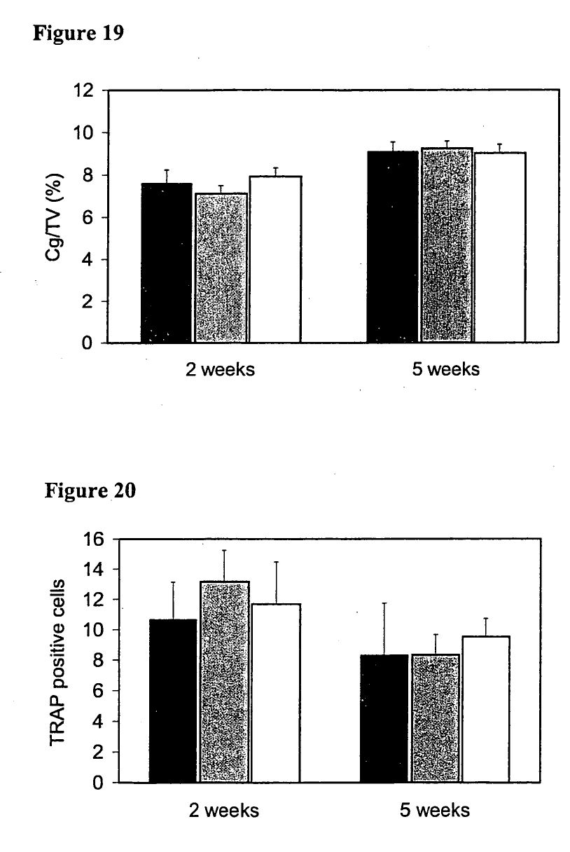

Safranin O staining was used to determine whether there was an increase in cartilage production within the callus in response to HS supplementation. Paraffin embedded sections were stained with Safranin-O to assess the percentage of cartilage formed within the total callus (Cg/TV). The cartilage stains red from the safranin O, the nuclei stain blue from the haematoxylin and the bone stains green from the light green counterstain. Similar to trabecular bone formation measurements, the amount of cartilage within each callus (Cg) and the total callus volume (TV) were measured, and from these measurements, the percentage of cartilage within the total callus volume (Cg/TV) was determined. The results were averaged for 9 samples per group, and the results are presented as the mean±standard deviation (see FIG. 19).

The results demonstrated that Cg/TV measurements were equal for all 3 groups at both 2 and 5 weeks (see FIG. 19), suggesting that HS does not influence cartilage formation in healing fractures.

### Determination of the Osteoclast Number

To analyse the specifity of the effect of HS on bone healing, the number of osteoclasts was determined. Osteoclasts are cells originating from monocyte/macrophage lineage precursors that specialize in bone resorption.

Resin sections were stained with tartrate-resistant acid phosphatase (TRAP) staining to assess osteoclast number. Nine fields of view within the callus were taken for each sample at 20× magnification using an Olympus Bx51 microscope, DP70 camera and DPController software V1.1.1.65. Osteoclasts positive for TRAP and containing more than 2 nuclei were then counted by visual inspection using a grid-technique. The results were averaged for each group and presented as the mean±standard deviation.

Multinucleated osteoclasts were primarily observed along the bone/cartilage interface within the callus, with more osteoclasts detected at 2 weeks than at 5 weeks (p<0.05; FIG. 21). However, between the groups at each time point, no difference was observed although there was a trend toward there being more osteoclasts in the 5 μg HS group compared to the other groups. Hence, HS does not show an effect on the number of osteoclasts present.

It is understood that the invention described in detail herein is susceptible to modification and variation, such that embodiments other than those described herein are contemplated which nevertheless falls within the broad scope of the invention.

The disclosure of each patent and scientific document, computer program and algorithm referred to in this specification is incorporated by reference in its entirety.

TABLE I

| Disaccharide | GAG (%) |
|---|---|
| IdoA/GlcA-AMan_{R} | 24.2 |
| IdoA(2S)-AMan_{R} | 27.6 |
| GlcA-AMan_{R}(6S) | 9.7 |
| IdoA-AMan_{R}(6S) | 5.4 |
| IdoA(2S)-AMan_{R}(6S) | 23 |
| GlcA(2S)-AMan_{R} | 1.9 |
| GlcA-AMan_{R}(3S) | nd |
| GlcA-AMan_{R}(3,6S) | nd |
| UNKNOWN | 8.0 |

TABLE II

| Peak number | Disaccharide | GAG (%) |
|---|---|---|
| 1 | ΔHexUA-GlcNAc | 50.7 |
| 3 | ΔHexUA-GlcNSO₃ | 19.1 |
| 2 | ΔHexUA-GlcNAc(6S) | 4.7 |
| 7 | ΔHexUA(2S)-GlcNAc | 2.6 |
| 4 | ΔHexUA-GlcNSO₃(6S) | 2.8 |
| 5 | ΔHexUA(2S)-GlcNSO₃ | 9.1 |
| 8 | ΔHexUA(2S)-GlcNAc(6S) | 0 |
| 6 | ΔHexUA(2S)-GlcNSO₃(6S) | 5.8 |
| 9 | Unknown | 5.1 |

TABLE III

| | Adenoma | Carcinoma |
|---|---|---|
| UA-GlcNAc | 51 | 54 |
| UA-GlcNS | 16 | 14 |
| UA-GlcNAc(6S) | 12 | 16 |
| UA(2S)-GlcNAc | 2 | 2 |
| UA-GlcNS(6S) | 6 | 6 |
| UA(2S)-GlcNS | 10 | 4 |
| UA(2S)-GlcNAc(6S) | 0 | 0 |
| UA(2S)-GlcNS(6S) | 3 | 4 |
| % N-sulfation | 35 | 28 |
| % 2-O-sulfation | 15 | 10 |
| % 6-O-sulfation | 21 | 26 |
| No. sulfates/100 disaccharides | 73 | 61 |

TABLE IV

| | 2 weeks | | | 5 weeks | | |
|---|---|---|---|---|---|---|
| | Saline | 5 μg HS | 50 μg HS | Saline | 5 μg HS | 50 μg HS |
| AP | 7.24 ± 0.663 | 8.91 ± 0.89* | 6.70 ± 0.389 | 6.21 ± 1.10 | 6.342 ± 0.89 | 6.26 ± 0.802 |
| Lat | 7.51 ± 1.42 | 6.78 ± 1.02 | 7.45 ± 1.24 | 6.98 ± 1.22 | 6.79 ± 0.912 | 7.33 ± 0.984 |

What is claimed is:

1. A surgical implant, prosthesis or bioscaffold comprising isolated heparin sulphate obtained from bone, bone cell, bone precursor cell or stem cell.

2. The surgical implant, prosthetic or bioscaffold of claim 1 for use with hard tissue.

3. The surgical implant, prosthetic or bioscaffold of claim 2, wherein the hard tissue is bone.

4. The surgical implant, prosthesis or bioscaffold of claim 1 for the repair of dental damage.

5. A method of treating an animal in need of tissue repair comprising a step of administering a pharmaceutical composition comprising:
a) isolated heparin sulphate obtained from bone, bone cell, bone precursor cell or stem cell, and
b) a carrier diluent.

6. The method of claim 5, wherein the tissue is hard tissue and the repair of said hard tissue comprises the step of administering the pharmaceutical composition by coating or impregnating a surgical implant, prosthesis or bioscaffold of claim 1 before implantation.

7. The method of claim 5, wherein said animal is a mammal.

8. The method of claim 7, wherein the mammal is a human, bovine, pig, or rodent.

9. The use of isolated heparin sulphate obtained from bone, bone cell, bone precursor cell or stem cell for stimulating regeneration of tissue.

10. A process for stimulating regeneration of tissue comprising a step of applying isolated heparin sulphate obtained from bone, bone cell, bone precursor cell or stem cell to an area of the body in need of soft or hard tissue regeneration.

11. The process of claim 10, wherein the hard tissue is bone.


SEQUENCE LISTING (8 sequences)

SEQ ID NO: 1 - DNA, 22 bp, artificial sequence
  Primer
gataacgagatgccaccagagg

SEQ ID NO: 2 - DNA, 21 bp, artificial sequence
  Primer
tccacgtcggttctgttcttc

SEQ ID NO: 3 - DNA, 24 bp, artificial sequence
  Primer
ccaggtttctgatgaacagtatcc

SEQ ID NO: 4 - DNA, 22 bp, artificial sequence
  Primer
acttgactcatggctgcccttt

SEQ ID NO: 5 - DNA, 24 bp, artificial sequence
  Primer
acaaacaaccacagaaccacaagt

SEQ ID NO: 6 - DNA, 20 bp, artificial sequence
  Primer
gtctcggtggctggtagtga

SEQ ID NO: 7 - DNA, 24 bp, artificial sequence
  Primer
gagggcaataaggtagtgaacaga

SEQ ID NO: 8 - DNA, 24 bp, artificial sequence
  Primer
aagccatactggtttgatagctcg